SEQUENCE LISTING (25 sequences)

SEQ ID NO: 1 - DNA, 43 bp, synthetic construct
  misc_feature 1..43: note = oligonucleotide ON1
  source 1..43: mol_type = other DNA
agcaaatgtctagattatccctaacatttaaatgcaatgaaaa

SEQ ID NO: 2 - DNA, 10 bp, synthetic construct
  misc_feature 1..10: note = oligonucleotide ON2
  source 1..10: mol_type = other DNA
tccctttttt

SEQ ID NO: 3 - DNA, 28 bp, synthetic construct
  misc_feature 1..28: note = oligonucleotide ON3
  source 1..28: mol_type = other DNA
tctagattatcccttttttttttttttt

SEQ ID NO: 4 - DNA, 48 bp, synthetic construct
  misc_feature 1..48: note = oligonucleotide ON4
  source 1..48: mol_type = other DNA
gaagcaaatgtctagattatcccttttttttttttttttttttttttt

SEQ ID NO: 5 - DNA, 67 bp, synthetic construct
  misc_feature 1..67: note = oligonucleotide ON5
  source 1..67: mol_type = other DNA
gaagcaaatgtctagattatccctttttttttttttttttttttttttttttttgtttaaacattta

SEQ ID NO: 6 - DNA, 51 bp, synthetic construct
  misc_feature 1..51: note = oligonucleotide ON6
  source 1..51: mol_type = other DNA
agggggggagggagaggggtaccccctatccttagtcaccaccggccccct

SEQ ID NO: 7 - DNA, 38 bp, synthetic construct
  source 1..38: mol_type = other DNA
gggagaggggtacccccttttttttttttttttttttt

SEQ ID NO: 8 - DNA, 48 bp, synthetic construct
  source 1..48: mol_type = other DNA
ctttgactcccatggtggcgtacccccttttttttttttttttttttt

SEQ ID NO: 9 - DNA, 31 bp, synthetic construct
  source 1..31: mol_type = other DNA
tgttttgttttacccctttggtttttttttt

SEQ ID NO: 10 - DNA, 1332 bp, synthetic construct
  misc_feature 1..1332: note = plasmid PL1
  source 1..1332: mol_type = other DNA
gccgaattatattatttttgccaaataatttttaacaaaagctctgaagtcttcttcatttaaattcttagatgatacttcatctggaaaattgtcccaattagtagcatcacgctgtgtaatacgactcactatagggggtacgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggatcctagggccgcagctcgctttcttgctgtccaatttctattaaaggttcctttgttccctaagtccaactactaaactgggggatattatgaagggccttgagcatttggattctgcctaataaaaaacatttattttcattgcgtttagctcgctttcttgctgtccaatttctattaaaggttcctttgttccctaagtccaactactaaactgggggatattatgaagggccttgagcatttggattctgcctaataaaaaacatttattttcattgcatttaaatgtttaaacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagcaggtgggagggtacgttcgtgataaacaaac

SEQ ID NO: 11 - DNA, 1179 bp, synthetic construct
  misc_feature 1..1179: note = plasmid PL2
  source 1..1179: mol_type = other DNA
gcccctgcagccgaattatattatttttgccaaataatttttaacaaaagctctgaagtcttcttcatttaaattcttagatgatacttcatctggaaaattgtcccaattagtagcatcacgctgtgtaatacgactcactatagggggtacgccaccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagttggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccttggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaggatcctagggccgcagctcgctttcttgctgtccaatttctattaaaggttcctttgttccctaagtccaactactaaactgggggatattatgaagggccttgagcatttggattctgcctaataaaaaacatttattttcattgcgtttagctcgctttcttgctgtccaatttctattaaaggttcctttgttccctaagtccaactactaaactgggggatattatgaagggccttgagcatttggattctgcctaataaaaaacatttattttcattgcatttaaatgtttaaacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagcaggtgggagggtacgttcgtgataaacaaac

SEQ ID NO: 12 - DNA, 1137 bp, synthetic construct
  misc_feature 1..1137: note = plasmid PL3
  source 1..1137: mol_type = other DNA
ctgcagccgaattatattatttttgccaaataatttttaacaaaagctctgaagtcttcttcatttaaattcttagatgatacttcatctggaaaattgtcccaattagtagcatgccacgctgtgtaatacgactcactatagggataatctagacatttgcttctgacacaactgtgttcactagcaacctcaaacagacaccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagttggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccttggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaggatcctagggccgcagctcgctttcttgctgtccaatttctattaaaggttcctttgttccctaagtccaactactaaactgggggatattatgaagggccttgagcatttggattctgcctaataaaaaacatttattttcattgcgtttagctcgctttcttgctgtccaatttctattaaaggttcctttgttccctaagtccaactactaaactgggggatattatgaagggccttgagcatttggattctgcctaataaaaaacatttattttcattgcatttaaatgtttaaacaaaaaaaaaaaaaaaaaaaaaaaaaaaaaaaaaagcaggtgggagggtacgttcgtgataaacaaacggccgc

SEQ ID NO: 13 - DNA, 1667 bp, synthetic construct
  misc_feature 1..1667: note = plasmid PL4
  source 1..1667: mol_type = other DNA
cctgcagccgaattatattatttttgccaaataatttttaacaaaagctctgaagtcttcttcatttaaattcttagatgatacttcatctggaaaattgtcccaattagtagcatgccacgctgtgtaatacgactcactatagggggtacccctctccctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacacatatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagttggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccttggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaggatcctagggccgcagctcgctttcttgctgtccaatttctattaaaggttcctttgttccctaagtccaactactaaactgggggatattatgaagggccttgagcatttggattctgcctaataaaaaacatttattttcattgcgtttagctcgctttcttgctgtccaatttctattaaaggttcctttgttccctaagtccaactactaaactgggggatattatgaagggccttgagcatttggattctgcctaataaaaaacatttattttcattgcatttaaatgtttaaacaaaaaaaaaaaaaaaaaaaaaaaaaaaaaaaaaagcaggtgggagggtacgttcgtgataaacaaacggccgct

SEQ ID NO: 14 - DNA, 1111 bp, synthetic construct
  misc_feature 1..1111: note = plasmid PL5
  source 1..1111: mol_type = other DNA
cctgcagccgaattatattatttttgccaaataatttttaacaaaagctctgaagtcttcttcatttaaattcttagatgatacttcatctggaaaattgtcccaattagtagcatgccacgctgtgtaatacgactcactatagggggtaatgagtcctggactgaaacggactcatatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagttggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccttggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaggatcctagggccgcagctcgctttcttgctgtccaatttctattaaaggttcctttgttccctaagtccaactactaaactgggggatattatgaagggccttgagcatttggattctgcctaataaaaaacatttattttcattgcgtttagctcgctttcttgctgtccaatttctattaaaggttcctttgttccctaagtccaactactaaactgggggatattatgaagggccttgagcatttggattctgcctaataaaaaacatttattttcattgcatttaaatgtttaaacaaaaaaaaaaaaaaaaaaaaaaaaaaaaaaaaaagcaggtgggagggtacgttcgtgataaacaaacggccgct

SEQ ID NO: 15 - DNA, 1287 bp, synthetic construct
  misc_feature 1..1287: note = plasmid PL6
  source 1..1287: mol_type = other DNA
gcccctgcagccgaattatattatttttgccaaataatttttaacaaaagctctgaagtcttcttcatttaaattcttagatgatacttcatctggaaaattgtcccaattagtagcatgccacgctgtgtaatacgactcactatagggggtaggcgcgccagtccttctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaagcgactcggacatatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagttggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccttggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaggatcctagggccgcagctcgctttcttgctgtccaatttctattaaaggttcctttgttccctaagtccaactactaaactgggggatattatgaagggccttgagcatttggattctgcctaataaaaaacatttattttcattgcgtttagctcgctttcttgctgtccaatttctattaaaggttcctttgttccctaagtccaactactaaactgggggatattatgaagggccttgagcatttggattctgcctaataaaaaacatttattttcattgcatttaaatgtttaaacaaaaaaaaaaaaaaaaaaaaaaaaaaaaaaaaaagcaggtgggagggtacgttcgtgataaacaaacggccgct

SEQ ID NO: 16 - DNA, 1667 bp, synthetic construct
  misc_feature 1..1667: note = plasmid PL7
  source 1..1667: mol_type = other DNA
gcccctgcagccgaattatattatttttgccaaataatttttaacaaaagctctgaagtcttcttcatttaaattcttagatgatacttcatctggaaaattgtcccaattagtagcatgccacgctgtgtaatacgactcactatagggggtacccctctccctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacacatatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagttggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccttggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaggatcctagggccgcagctcgctttcttgctgtccaatttctattaaaggttcctttgttccctaagtccaactactaaactgggggatattatgaagggccttgagcatttggattctgcctaataaaaaacatttattttcattgcgtttagctcgctttcttgctgtccaatttctattaaaggttcctttgttccctaagtccaactactaaactgggggatattatgaagggccttgagcatttggattctgcctaataaaaaacatttattttcattgcatttaaatgtttaaacaaaaaaaaaaaaaaaaaaaaaaaaaaaaaaaagagacggggtacgttcgtgatacggccgcttcgagcaga

SEQ ID NO: 17 - DNA, 1092 bp, synthetic construct
  source 1..1092: mol_type = other DNA
gcccctgcagccgaattatattatttttgccaaataatttttaacaaaagctctgaagtcttcttcatttaaattcttagatgatacttcatctggaaaattgtcccaattagtagcatcacgctgtgtaatacgactcactatagggggtacgccaccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagttggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccttggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaggatcctagggccgcagctcgctttcttgctgtccaatttctattaaaggttcctttgttccctaagtccaactactaaactgggggatattatgaagggccttgagcatttggattctgcctaataaaaaacatttattttcattgcgtttagctcgctttcttgctgtccaatttctattaaaggttcctttgttccctaagtccaactactaaactgggggatattatgaagggccttgagcatttggattctgcctaataaaaaacatttattttcattgcatttaaatgtttaaacaaaaaaaaaaaaaaaaaaaaaaaaaaaaaaaaaagcaggtgggagggtacgttcgtgataaacaaacggccgct

SEQ ID NO: 18 - DNA, 1678 bp, synthetic construct
  source 1..1678: mol_type = other DNA
gcccctgcagccgaattatattatttttgccaaataatttttaacaaaagctctgaagtcttcttcatttaaattcttagatgatacttcatctggaaaattgtcccaattagtagcatgccacgctgtgtaatacgactcactataggggtaaaacaaaacacacccctctccctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacacatatggtgtctaagggggaggagctgttcaccggcgtggtgcccatcctggtggagctggacggcgacgtgaacggccacaagttcagcgtgagcggggagggcgagggcgacgccacctacggcaagctgaccctgaagttcatctgcacaacggggaagctgcccgtgccctggcccaccctggtgaccaccctgacctacggcgtgcagtgcttcagccggtaccccgaccacatgaagcagcacgacttcttcaagagcgccatgcccgaggggtacgtgcaggagcggaccatcttcttcaaggacgacgggaactacaagacccgcgccgaggtgaagttcgagggggacaccctggtgaaccgcatcgagctgaagggcatcgacttcaaggaggacggcaacatcctggggcacaagctggagtacaactacaacagccacaacgtgtacatcatggccgacaagcagaagaacggcatcaaggtgaacttcaagatccggcacaacatcgaggacggcagcgtgcagctggccgaccactaccagcagaacacccccatcggcgacggccccgtgctcctgcccgacaaccactacctgtccacccagagcgccctgagcaaggacccaaacgagaagcgggaccacatggtcctgctggagttcgtgaccgccgccggcatcaccctgggcatggacgagctgtacaagcgctcccgcgacatcagccacggcttccctcccgccgtggccgcccaggacgacggcaccctgcccatgagctgcgcccaggagagcggcatggaccggcacccggccgcctgcgccagcgcccggatcaacgtgtgatagggatccaacaaaaaaaacaaaaaaaaaaccaaagagacgcggccgcttcgagcagacatgataagatacatt

SEQ ID NO: 19 - DNA, 1793 bp, synthetic construct
  source 1..1793: mol_type = other DNA
gcccctgcagccgaattatattatttttgccaaataatttttaacaaaagctctgaagtcttcttcatttaaattcttagatgatacttcatctggaaaattgtcccaattagtagcatgccacgctgtgtaatacgactcactatagggggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaacccccctctccctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacacatatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagttggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccttggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagcggccgcttcgagcagacatgataagatacattgatgatc

SEQ ID NO: 20-21 - skipped (2 entries)

SEQ ID NO: 22 - DNA, 16 bp, synthetic construct
  source 1..16: mol_type = other DNA
aaaaaaaaagcaggtg

SEQ ID NO: 23-24 - skipped (2 entries)

SEQ ID NO: 25 - DNA, 21 bp, synthetic construct
  source 1..21: mol_type = other DNA
ccttcagctcgatgcggttca

(19) United States
(12) Patent Application Publication
(10) Pub. No.: US 2026/0327253 A1
(43) Pub. Date: Oct. 22, 2026

(54) CIRCULAR RNA ANALOGS, PREPARATION AND APPLICATION THEREOF

(71) Applicant: Siec Badawcza Lukasiewicz PORT Polski Osrodek Rozwoju Technologii, Wroclaw (PL)

(72) Inventors: Adam Mamot, Warszawa (PL); Malgorzata Wasinska-Kalwa, Warszawa (PL); Karol Czubak, Skarzysko Koscielne (PL); Katarzyna Frankowska, Warszawa (PL); Mikolaj Chrominski, Warszawa (PL); Joanna Kowalska, Warszawa (PL); Jacek Jemielity, Warszawa (PL); Dominika Nowis, Warszawa (PL); Jakub Golab, Warszawa (PL)

(21) Appl. No.: 19/485,385

(22) PCT Filed: May 20, 2024
(86) PCT No.: PCT/EP2024/063843

§ 371 Date: Nov. 18, 2025

(30) Foreign Application Priority Data
May 18, 2023 (EP) 23461592.0

(51) Int. Cl. C07H 21/02; A61K 48/00; A61K 49/00; C07H 1/00

(52) U.S. Cl. CPC C07H 21/02; A61K 48/005; A61K 48/0091; A61K 49/00; C07H 1/00; A61K 2123/00

(57) ABSTRACT

The presented invention relates to a novel class of nucleic acids, which have circular topology and contain at least one unnatural internucleotide linkage. The main objects of the disclosed invention are analogs of circular RNA (circRNA) that are obtained by the synthesis of 5′-modified precursors (pre-circRNAs) and their subsequent circularization via a chemical reaction.

### SUMMARY OF THE INVENTION

The presented invention relates to a novel class of nucleic acids, which have circular topology and contain at least one unnatural internucleotide linkage. The main objects of the disclosed invention are analogs of circular RNA (circRNA) that are obtained by the synthesis of 5′-modified precursors (pre-circRNAs) and their subsequent circularization via a chemical reaction. The circularization occurs due to the presence of two chemically reactive functional groups, one at RNA 5′ end and another at the 3′ end. The functional group at the 5′ end is incorporated co-transcriptionally, whereas the reactive group at the 3′ end is incorporated post-transcriptionally. The invention encompasses RNAs of different lengths (from 2 nt to 20000 nt), including messenger RNAs (RNAs encoding a protein). The circRNAs encoding a protein according to this invention undergo translation with an efficiency higher than chemically unmodified circRNAs of the same sequence. Methods of preparation of circRNA analogs, methods of preparation of corresponding pre-circRNAs, methods of preparation of a protein or peptide, and application of the disclosed circRNA analogs in the context of gene therapies and vaccinations are also disclosed.

### FIELD OF THE INVENTION

The presented invention relates to RNA molecules, methods of producing circular RNA molecules, methods of producing a protein or peptide in vitro, in cells, or living organisms, and methods of treating or preventing a disease.

### BACKGROUND OF THE INVENTION

Despite its inherent fragility, RNA is one of the most potent substances in the context of therapeutic gene delivery. The potency of therapeutic messenger RNA (mRNA) stems from both advanced methods of delivery and chemical modification of the mRNA itself. Both in serum and in the cytosol, mRNA is primarily targeted by exonucleases (e.g. decapping complexes, deadenylases, exosomes).² Circularization is one of the most protective modifications of mRNA, as it prevents exonucleolytic digestion.³

Currently known methods of RNA circularization are based on chemical reactions^{4, 5}, enzymatic reactions⁶⁻⁹, and reactions catalyzed by catalytic sequences embedded in circRNA precursors¹⁰⁻¹³ (pre-circRNA).

The reported methods of circularization that are based on chemical reactions rely on the use of short RNA sequences, chemically synthesised via phosphoramidite method, such as 3′ phosphorylated RNA or 3′-amino RNA, which undergo condensation induced by cyanogen bromide (BrCN) or 1-ethyl-3-(3-dimethylaminopropyl)carbodiimide (EDC).^{4, 5} The phosphoramidite solid phase synthesis of nucleic acids is a robust method of production of modified and unmodified oligonucleotides, however it is not practical for the production of long (>100 nt) RNA sequences. In consequence, circRNA obtained with these methods encode relatively short sequences and has limited use in the context of RNAs encoding a protein or peptide, which are usually significantly longer.

Moreover, RNA circularization can be carried out with ligases, especially T4 RNA ligase I and T4 RNA ligase II.^{6, 7, 14} Both enzymes require 5′ monophosphorylated precursors, which are first adenylated and subsequently ligated with the 3′ OH of the ribose at the 3′ end of a different or the same RNA strand.¹⁵ Both enzymes are capable of intramolecular ligation (circularization) of RNA (if reaction occurs with the 3′ end of the same RNA strand); however, they have been found to be strongly dependent on target RNA length and sequence.^{11, 12, 14, 16} This is detrimental, as it difficult to predict or improve performance of such enzymes.

Litke et al. have shown the Twister-optimized RNA durable overexpression (Tornado) system for RNA circularization.⁸ Tornado-expressed transcripts contain an RNA of interest flanked by Twister ribozymes that spontaneously undergo autocatalytic cleavage. This leaves 5′-hydroxyl and 2′,3′-cyclic phosphate termini, which are then ligated by the RNA ligase RtcB.

The so-far-reported methods based on catalytic nucleic acid activity require sequence design and development, therefore cannot be used on pre-circRNA of any given sequence. Furthermore, pre-circRNA's ability to undergo circularization can be hindered by the presence of chemical modifications, such as natural and synthetic nucleosides and nucleobases.¹²

WO/2021/113777 discloses circular RNAs and transfer vehicles, along with related compositions and methods of treatment. Pharmaceutical compositions comprising such circular RNAs, as well as precursor RNAs and materials useful in producing the precursor or circular RNAs, have been also disclosed in cited document.

WO/2019/236673 describes a vector for making circular RNA, allowing the production of a circular RNA that is translatable or biologically active inside eukaryotic cells.

EP4008336 discloses a recombinant nucleic acid molecule of the transcriptional circular RNA, a recombinant expression vector, pre-circularized RNA, circular RNA, a recombinant host cell, a pharmaceutical composition and a protein preparing method. The transcription product of the recombinant nucleic acid molecule is a circular RNA which containing specific IRES element.

US20150079630 discloses cyclic RNA and a method for production thereof which comprises the use of T4 DNA ligase.

None of the previously reported methods provide analogs of circular RNA (circRNA) that are obtained by the synthesis of 5′-modified precursors (pre-circRNAs) and their subsequent circularization via a chemical reaction in the absence of protein enzymes or catalytic nucleic acid sequences.

### SUMMARY

The present disclosure provides novel analogs of circRNAs that can be prepared from pre-circRNAs via a chemical reaction (chemical circularization of RNA), regardless of circRNA sequence, length or size. The proposed chemical circularization of RNA is compatible with the presence of other chemical modifications of the RNA body, such as natural and synthetic nucleosides and nucleobases, or fluorescent tags.

The present disclosure provides a circRNA analog prepared by in vitro transcription reaction followed by a subsequent chemical circularization reaction, wherein said circularization is obtained in the absence of protein enzymes or catalytic nucleic acid sequences.

The present disclosure provides a circRNA analog, that contains a synthetic linear or branched linker and possibly an internal ribosomal entry site sequence.

The present disclosure provides a circRNA analog according to formula 1:

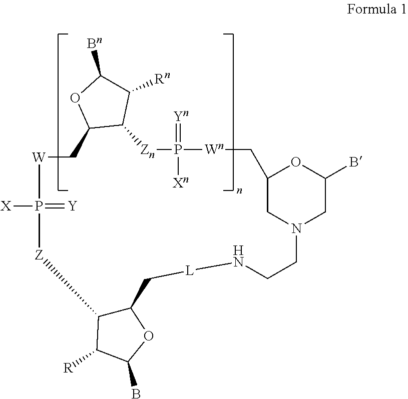

wherein:
n is any integer with a value from 1 to 20 000;
X and each of Xⁿ is independently OH, BH₃, NH₂, or SH;
Y and each of Yⁿ is independently O or S;
Z and each of Zⁿ is independently O, S or NH;
W and each of Wⁿ is independently O, S or NH;
R and each of Rⁿ is independently H, OH, alkyl, O-alkyl or halogen;
B, B′ and each of Bⁿ is independently a natural, modified or unnatural nucleoside base;
L is a linear or branched linker.

In some embodiments, the linker L is a single bond or a linker according to one among of formulas 2-7:

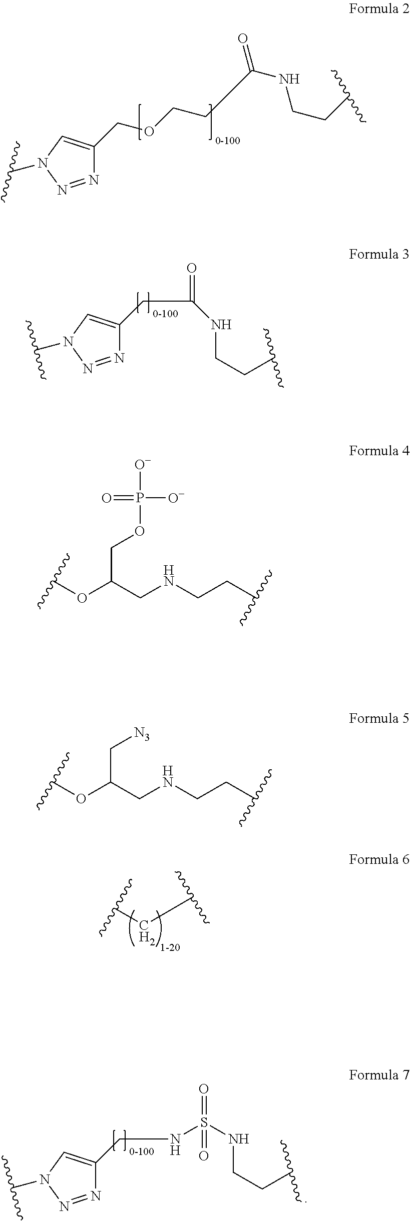

In some embodiments, the linker L comprises a detectable tag or label.

The present disclosure provides a method of synthesis of circRNA analogs as defined above, characterized in that a circRNA analog precursor (formula 8)

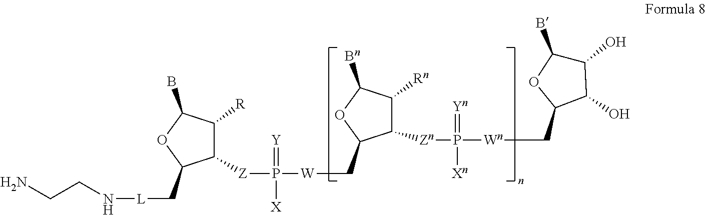

wherein:
n=1-20 000;
X and each of Xⁿ is independently OH, BH₃, NH₂, or SH;
Y and each of Yⁿ is independently O or S;
Z and each of Zⁿ is independently O, S or NH;
W and each of Wⁿ is independently O, S or NH;
R and each of Rⁿ is independently H, OH, alkyl, O-alkyl or halogen;
B, B′ and each of Bⁿ is independently a natural, modified or unnatural nucleoside base;
L is a linear or branched linker;
is first oxidized during step (i) of the method, yielding a dialdehyde derivative (formula 9),

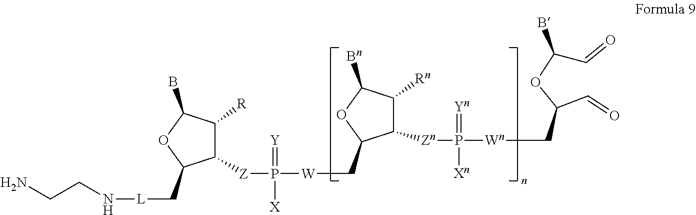

wherein:
n=1-20 000;
X and each of Xⁿ is independently OH, BH₃, NH₂, or SH;
Y and each of Yⁿ is independently O or S;
Z and each of Zⁿ is independently O, S or NH;
W and each of Wⁿ is independently O, S or NH;
R and each of Rⁿ is independently H, OH, alkyl, O-alkyl or halogen;
B, B′ and each of Bⁿ is independently a natural, modified or unnatural nucleoside base;
L is a linear or branched linker;
which is next incubated in reductive environment during step (ii) of the method, yielding the desired circRNA analog.

In some embodiments of provided method, the linker L the linker L is a single bond or a linker according to one among of formulas 2-7:

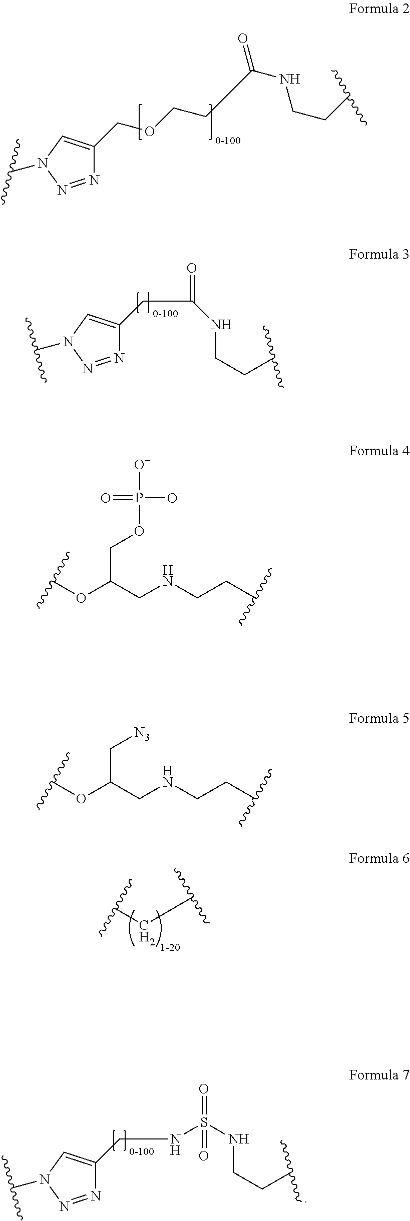

In some embodiments, step (i) and step (ii) are carried out in one reactor. In some embodiments, step (i) of the method is carried out in the presence of NaIO₄, preferably at a concentration below 10 mM, at a temperature below 60° C. In some embodiments, step (ii) of the method is carried out in a buffered solution, preferably at pH 5-8, in presence of NaBH₃CN below 100 mM. In some embodiments, step (i) or step (ii) of the method are carried out in presence of a natural, modified or unnatural nucleic acid oligomer that is complementary to a RNA precursor sequence. In some embodiments, the circRNA analog is isolated from the reaction mixture by a known method of RNA isolation, such as precipitation in alcohol, size exclusion chromatography, gel electrophoresis, or high-performance liquid chromatography (HPLC).

The present disclosure provides a use of a circRNA analog described above in protein production in an in vitro setup.

The present disclosure provides a circRNA analog described above for use in a therapeutic or diagnostic method, in particular for protein production in an in vivo setup.

The present disclosure provides a pharmaceutical composition comprising of a circRNA analog described above.

### Definitions

Unless defined otherwise, all terms used herein have the same meaning as are commonly understood by one of skill in the art to which this invention belongs.

The terms “nucleic acid” and “nucleic acid polymer” refer to a polymeric substance composed of ribo or deoxyribo derivatives and of naturally occurring or synthetic nucleoside monomers, including noncanonical linkages and any degree of polymerization. These phrases include, but are not limited to natural or synthetic RNA and DNA, as well as nucleic acids composed of phosphorothioate, boranophosphate, selenophosphate, and phosphonate moieties, morpholino nucleic acids (MNA), locked nucleic acid (LNA), peptide nucleic acid (PNA), and threose nucleic acid (TNA).

The terms “modification”, “modifying group”, and “modified” in the context of nucleic acids refer to any chemical moiety that may be attached to a nucleic acid molecule. This includes, but is not limited to attachment of the modifying group to the sugar, nucleobase, internucleotide phosphate, or 5′-5′-oligophosphate of a nucleic acid molecule.

The term “internucleotide linkage” refers to a covalent or non-covalent bond or bonds that connect two monomers of a nucleic acid polymer.

The terms “nucleoside base”, “nucleobase” and “nitrogenous base” refer to a part of nucleic acid molecules. The referred nucleobases may be involved in base pairing, base stacking, and other inter- and intramolecular interactions. The referred nucleobases include, but are not limited to natural, modified, unnatural or synthetic purine (such as adenine, guanine, hypoxanthine, xanthine, N7-methylguanosine, N6-methyladenosine, N2-methyladenosine) and pyrimidine (such as cytosine, thymidine, uracil, dihydrouracil, 5-methylcytosine, 5-hydroxymethylcytosine, pseudouridine, methylpseudouridine, nicotinamide) nucleobases, as well as 2-aminopurine, 2,6-diaminopurine, flavin, C8-substituted adenine, C8-substituted guanine, C7-substituted 7-deazaadenine, C7-substituted 7-deazaguanine, C5-substituted uridine, C5-substituted cytosine, di- and tricyclic nucleobases.

The terms “nucleic acid sequence”, “nucleotide sequence”, “nucleoside sequence”, “nucleobase sequence” and “sequence” refer to the order and composition of a series of nucleobases within a nucleic acid molecule.

The terms “nucleobase pair” and “base pair” refer to a fundamental unit of double-stranded nucleic acids consisting of two nucleobases bound to each other by hydrogen bonds. The term “base pairing” refers to specific hydrogen bonding between nucleobases, that can be canonical (Watson-Crick base paring) or non-canonical (e.g. wobble base paring, Hoogsten base paring).

The terms “hybridization” and “hybridize” refer to interaction of two or more nucleic acid strands. It is characterized by that, that under appropriate conditions distinct nucleic acid strands maintain a sequence-dependent structure (e.g. a double helix, a triple helix, an I-motif, a G-quadruplex).

The term “complementary” refers to relation between sequences of two or more strands of a nucleic acid or nucleic acids. The complementary sequences or strands are characterised by that they undergo hybridization and form a sequence-dependent structure. The complementarity can be full or partial.

The terms “linear” and “circular” in context of nucleic acids refer to the topology of a nucleic acid molecule. Linear nucleic acids are linear polymers with no linkage between 5′ and 3′ ends. Circular nucleic acids are polymers with natural or modified linkage between 5′ and 3′ ends.

The terms “circularization” and “cyclization” refer to a process of transformation of a linear nucleic acid polymer to a circular nucleic acid polymer.

The terms “circRNA precursor” and “pre-circRNA” refer to a linear nucleic acid polymer that can undergo circularization leading to a circular nucleic acid polymer.

The term “linker” in context of circular nucleic acids refers to any natural or synthetic combination of chemical moieties that covalently join 5′ and 3′ ends of the polymer. The term “linker” includes, but is not limited to any linear or branched combination of alkyl, alkenyl, alkynyl, ether, ester, amide, amine, carbamate, triazol, phosphoester, amidophosphate, or phosphonate moieties. In particular it can be selected among of following structures:

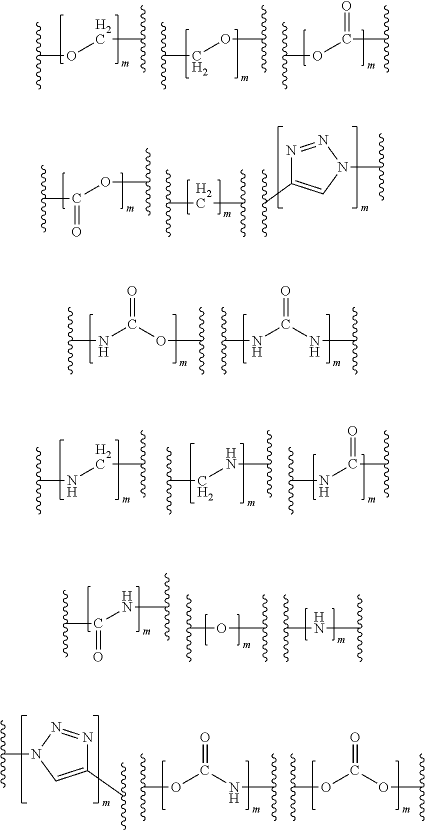

wherein m is a natural number from 1 to 100.

The term “cap analog” refers to any compound that contains natural or modified 5′-5′-oligophosphate linking a natural or modified nucleosides, nucleotides, or nucleic acids. The term “Cap analog” refers to canonical, as well as non-canonical cap structure and includes, but is not limited to Cap0, Cap1, Cap2, TMG-cap, NAD-cap, and FAD-cap, as well as cap structures modified within oligophosphate bridge, ribose, or nucleobases. In particular, it can be selected among of structures described in any document cited in subject description, especially: WO/2009/149253, WO/2008/157688, WO/2011/015347, WO/2022/191725, WO/2018/015845, WO/2014/093627, WO/2021/162566, WO/2021/162567 and WO/2017/130151.

The terms “internal ribosomal entry site” and “IRES” refer to RNA sequences that are characterised by their ability facilitate cap-independent initiation of translation by interaction or binding cellular proteins involved in formation of ribosomes and translation. The term “IRES sequence” includes, but is not limited to Encephalomyocarditis virus (EMCV) IRES, Coxsackievirus B3 (CVB3) IRES, respiratory syncytial virus (RSV) m⁶A methylation sites, viral IRES sequences, human IRES sequences, m⁶A methylation sites, sequences containing m⁶A modification, eIF4G aptamers, and artificial IRES sequences.^{11, 12, 22, 24}

The terms “transcription” or “transcription reaction” refer to methods for enzymatic synthesis of RNA that is complementary to a DNA template. Transcription can occur in cells, in cellular systems or in artificial setups (as in in vitro transcription reaction). Transcription is carried out in presence of a DNA-dependent RNA polymerase, ribonucleoside triphosphates (NTPs), and a DNA template. During the reaction, the sequence of the DNA template is copied and amplified in form of RNA molecules. Optionally, the transcription can be carried out in the presence of additional reagents, such as transcription initiators, transcription terminators, or modified nucleoside triphosphates.

The terms “label” and “detectable label” refer to any compound, particle, or any combination of compounds and/or particles, that may be directly or indirectly detected. The label can be associated with a target molecule by covalent and/or non-covalent interactions. The label can be a radioisotope, NMR label, spin label, a chromophore, a fluorophore, a hapten. The preferred label includes but is not limited to ²H, ¹³C, ¹⁵C, ³²P, ¹⁹F, cyanines, fluoresceines, rhodamines, coumarines, perylenes, naphthalimides, nitro dyes, azo dyes, BODIPY, biotin, desthiobiotin, folic acid and N-acetylgalactosamine.

The terms “periodate oxidation”, “cis-diol oxidation” and “ribose oxidation” refer to a process of cleavage of carbon-carbon bonds of vicinal cis-diols present in the structure of a natural or synthetic nucleic acids leading to a dialdehyde derivative of a nucleic acid.

The terms “reductive amination” and “amination” in the context of nucleic acid modification or circularization refer to amination reaction involving a dialdehyde derivative of a nucleic acid.

The term “reductive environment” refers to conditions under which chemical reduction reactions occur. The referred reductive environment includes but is not limited to the presence of sodium cyanoborohydride, sodium borohydride, pyridine borane, or sodium ascorbate.

The terms “halo” and “halogen” refer to all halogens, such as chloro, fluoro, bromo, and iodo groups.

The term “alkyl” refers to a single bond chain of hydrocarbons ranging from 1 to 20 carbon atoms and includes but is not limited to methyl, ethyl, propyl, isopropyl, n-butyl, sec-butyl, isobutyl, tert-butyl, pentyl, isoamyl, hexyl, octyl, and dodecanyl groups.

The term “O-alkyl” denotes the group-OR, where R is an alkyl, and includes but is not limited to O-methoxy, O-ethoxy, O-propoxy, O-isopropoxy, O-butoxy, O-isobutoxy, and O-pentoxy groups.

The terms “ethylenediamine motif”, “EDA motif”, “EDA group”, and “EDA” refer to a chemical motif of R—CH₂—NH—(CH₂)₂—NH₂, where R is any substituent. Structure of the EDA motif may as well be recognised in an ionic form or in a form of a salt.

The term “reactive functional group” refers to any functional group that is incorporated at the RNA 5′ or 3′ end that can react with another (same or different) reactive group in a selective manner in presence of other (natural or unnatural) chemical moieties contained within structure of the circRNA precursor. The representative pairs of 5′ and 3′ reactive groups which are reactive towards each other, include but are not limited to EDA and dialdehyde, hydrazide and dialdehyde, azide and alkyne, azide and cyclooctyne derivative, amino and carboxylic group, amino and fluorosulfonyl group, azide and phosphine, etc.

The terms “nucleic acids alcohol precipitation”, “nucleic acids precipitation”, and “alcohol precipitation” refer to the process of nucleic acid isolation. It is a technique to de-salt and concentrate nucleic acids from aqueous solutions. Addition of polar organic solvents (e. g. ethanol, isopropanol, acetone) and salts (e. g. sodium acetate, ammonium acetate, lithium chloride, sodium perchlorate, lithium perchlorate) to nucleic acid solution causes the precipitation of nucleic acids.

The terms “polyacrylamide gel electrophoresis” and “PAGE” refer to a technique used for separation of biological macromolecules according to their electrophoretic mobility. Electrophoretic mobility is a function of the mass, conformation, topology, and charge of the molecule. When PAGE is performed in denaturing conditions, it provides information on the composition of the nucleic acid species in the sample.

The terms “reversed-phase high pressure chromatography”, “RP-HPLC, and “HPLC” refer to a technique for separation, identification, quantification, and isolation of components of a sample mixture. It relies on pumps constituting a flow of pressurized liquid mobile phase (a mixture of solvents and solutions) through a solid matrix of the stationary phase. Components of the sample interact differently with mobile and stationary phases and are in equilibrium between adsorbed and solubilized states. This is facilitated by a combination of hydrophobic, dipole-dipole and ionic interactions. As chromatography progresses, the analytes are resolved based on their elution velocity, can be detected by their molecular properties, and collected into separate fractions (peaks). The elution velocity of individual components of the sample is parameterized by the retention time of the fraction and the peak shape. The active component of the stationary phase is a monolith or particle solid resin of organic and/or inorganic composition, such as silica, poli(styren-co-divinylbenzen), or other polymers. The mobile phase is an aqueous or organic solution or a mixture of aqueous and organic solutions. The chromatography may be performed in ion-pair mode, in elevated temperatures, and for analytical as well as preparative purposes.

The terms “translation in vitro”, “protein synthesis in vitro”, and “protein expression in vitro” refer to a process characterised as an enzymatic production of polypeptides based on sequence of RNA or RNA analog in an artificial system. An in vitro system includes but is not limited to artificial reaction mixture, cellular lysates, nuclear lysates, cell cultures, and tissue cultures.

The terms “translation in vivo”, “protein synthesis in vivo”, and “protein expression in vivo” refer to a process characterised as an enzymatic production of polypeptides based on sequence of RNA or RNA analog in a natural system. An in vivo system includes but is not limited to whole organisms, organs, cellular and tissue transplants of human, animal, plant, fungal, or bacterial origin.

The terms “therapeutic protein” or “therapeutic proteins” refer to a polypeptide molecule that may be utilized in the course of therapy or medical studies. The classes of therapeutic proteins include but are not limited to antigens, antibodies, immunogenic proteins, fluorescent proteins, reporter proteins, protein hormones, membrane proteins, transcription factors, regulatory proteins, systems for gene editing and systems for gene regulation.

The terms “therapeutic gene” or “therapeutic genes” refer to a sequence of a nucleic acid containing an open-reading frame encoding the amino acid sequence of the therapeutic protein. Therapeutic genes may consist of but are not limited to sequences encoded in natural or synthetic viral DNA or RNA, plasmid vectors, RNA, mRNA or circRNA.

The term “pharmaceutical composition” refers to a composition of substances comprising of active substances and helper substances, such as pharmaceutically acceptable solvents and/or carriers. Pharmaceutical composition may be formulated as a solid, liquid, aqueous solution, aqueous emulsion, suspension, lipid nanoparticles, liposomes, or liposomal suspension. The term pharmaceutical composition includes but is not limited to compositions designed for expression of a therapeutic genes and proteins in in vitro and in vivo setup.

The publications cited in the description and the references given therein are hereby incorporated as references.

### BRIEF DESCRIPTION OF THE DRAWINGS

The following detailed description of the embodiments of the invention will be better understood when read in conjunction with the appended drawings. For the purpose of illustrating the invention, there are shown in the drawings embodiments, which are presently exemplified. It should be understood, however, that the invention is not limited to the precise arrangement and instrumentalities of the embodiments shown in the drawings.

FIG. 1 shows the general concept of the preparation of circRNA analogs. A) First, the precursor of circRNA analog (FG-linker-RNA) is synthesized during an in vitro transcription (IVT) reaction. Due to the presence of a transcription initiator (FG-linker-AG), the transcribed RNA contains a reactive functional group (FG) at the 5′ end. Next, the 3′ end reactive functional group is incorporated into RNA post-synthetically, by either enzymatic reaction or preferably chemical reaction. The 5′ and 3′ reactive functional groups are reactive towards each other either spontaneously or in the presence of a specific trigger. B) As an example, a precursor of circRNA analog (EDA-linker-RNA) is synthesized during IVT. Due to the presence of a transcription initiator EDA-linker-AG, the transcribed RNA contains an ethylenediamine (EDA) moiety at the 5′ end. The precursor is first chemically modified at the 3′ end by treatment with periodate. This causes selective oxidation of the 3′ ribose, which is prone to nucleophilic attack by the EDA moiety. Finally, the addition of a reductive agent (i. e. NaBH₃CN) leads to an irreversible covalent linkage of RNA's 5′ and 3′ ends.

FIG. 2 shows that chemical circularization results in higher yields and product homogeneity than enzymatic circularization. A) The 5′-modified RNA (pre-circRNA2) was used as a precursor for chemical circularization. As a reference, 5′-monophosphorylated RNA of the same sequence (pre-circRNA1) was used as a substrate in enzymatic circularization catalyzed by T4 RNA ligase I. The chemical circularization results in higher yield of the desired circularized product; B) The 5′-modified RNA (pre-circRNA21) was used as a precursor for chemical circularization. As a reference, 5′-monophosphorylated RNA of the same sequence (pre-circRNA20) was used as a substrate in enzymatic circularization catalyzed by T4 RNA ligase I. The chemical circularization results in higher yield of the desired circularized product and notably lower content of side-products (linear dimer, circular dimer). The substrates and products of chemical and enzymatic reactions were analyzed by PAGE (6% AA, AA:MBAA=19:1, 7 M Urea, 1×TBE). The yields of the reactions were determined by densitometry. M refers to linear RNA length marker. HMW refers to linear RNA of length ranging from 1500 nt to 6000 nt.

FIG. 3 shows chemical circularization of long RNA molecules results in higher yield and less side-products than ribozymatic circularization (permuted intron-exon (PIE) splicing reaction). The splicing reaction was conducted using a self-splicing RNA precursor (pre-circRNA22), while the recircularization reaction was performed at increasing concentrations of mRNA. The precursor for chemical circularization included 5′-modified long RNAs (pre-circRNA19, pre-circRNA10, pre-circ RNA18). The substrates and products of chemical and self-splicing reactions were analyzed by PAGE (6% AA, AA:MBAA=37.5:0.5, 7 M Urea, 1×TBE). The yields of the reaction were determined by densitometry. M refers to linear RNA length marker. HMW refers to linear RNA of length ranging from 1500 nt to 6000 nt.

FIG. 4 shows an RNase R-mediated removal of linear RNA in the presence of circular RNA. Samples of a linear precursor of circRNA analog (pre-circRNA2) and products of chemical circularization (a mixture of pre-circRNA2 and circRNA2) were treated with a processive 3′ exonuclease (RNase R) that selectively hydrolyses linear RNA species. After RNase R treatment the samples were analyzed by PAGE (6% AA, AA:MBAA=38:1, 7 M Urea, 1×TBE). M refers to linear RNA length marker. HMW refers to linear RNA of length ranging from 2000 nt to 6000 nt.

FIG. 5 shows a RNase H digestion assay to confirm the circular topology of chemically circularized RNAs. Samples of a circRNA analog (circRNA2) and its linear precursor (pre-circRNA2) underwent DNA-dependent endonucleolytic cleavage catalyzed by RNase H and subsequent PAGE. In presence of targeting DNA oligonucleotide, the circRNA analog is nicked. As a result, nicked circRNA exhibits electrophoretic mobility of a linear precursor. On the other hand, cleavage of the linear precursor leads to two RNA molecules of lower size. PAGE conditions: 6% AA, AA:MBAA=38:1, 7 M Urea, 1×TBE. M refers to linear RNA length marker. HMW refers to linear RNA of length ranging from 2000 nt to 6000 nt.

FIG. 6 shows a polyacrylamide gel electrophoresis of circRNA-influence of gel crosslinking degree. Samples of a RNA length marker, a linear precursor of circRNA analog (pre-circRNA2), and products of chemical circularization (a mixture pre-circRNA2 and circRNA2) were analyzed by PAGE. Two types of 6% polyacrylamide gels were used in parallel: one with higher crosslinking degree (AA:MBAA=19:1, 7 M Urea, 1×TBE) and one with lower crosslinking degree (AA:MBAA=38:1, 7 M Urea, 1×TBE). The migration of circRNA is slower than that of linear RNA. Electrophoretic mobility differences are pronounced in gel with a higher crosslinking degree. HMW refers to linear RNA of length ranging from 2000 nt to 6000 nt.

FIG. 7 shows an optimization of RNA conformation using complementary DNA oligonucleotides. A) Prediction of minimum free energy structure of RNA5 sequence generated with RNAfold web server. B) Prediction of tertiary structure of an RNA sequence comprising flanking regions of RNA5 sequence generated with RNAComposer web server. The predictions indicate, that 3′ poliA sequence may distance 5′ and 3′ ends and hinder any reaction between them. Fluorescence spectra of Cy5-RNA5-Cy3 probe recorded at 4° C. (C) or 20° C. (D) before (w/o ON) or after hybridization with of one of the complementary DNA oligonucleotides (ON2-ON5). Emergence of 660 nm emission band indicates the occurrence of the FRET phenomenon facilitated by decreased distance between 5′ and 3′ ends.

FIG. 8 shows the influence of complementary DNA oligonucleotides on chemical circularization. Pre-circRNA10 was used as a precursor for chemical circularization. Before the oxidation step, the precursor was annealed with one of the complementary DNA oligonucleotides (ON2-ON5). After the reaction, the samples were analyzed by PAGE (6% AA, AA:MBAA=38:1, 7 M Urea, 1×TBE). As a reference, samples of precursor before reaction (NR) and products of reaction without complementary DNA (w/o ON) were included. M refers to linear RNA length marker. HMW refers to linear RNA of length ranging from 3000 nt to 6000 nt. The relative amounts of circular product (P), linear dimer (D), and unreacted substrate(S) were determined by densitometry.

FIG. 9 shows an attempted separation of linear and circular RNA using IE-HPLC (A), SEC-HPLC (B) and RP-HPLC (C). A sample containing a mixture of linear and circular RNA (pre-circRNA2+circRNA2) was chromatographed using three different modes and columns. As a reference, linear RNA (pre-circRNA2) was resolved in same conditions. IE-HPLC conditions: CIMac PrimaS™ 0.1 mL Analytical Column (2 μm); A—50 mM HEPES pH 7.0; B—200 mM sodium pyrophosphate, 50 mM HEPES pH 8.5; 0% B for 2 min, 0-100% B in 6 min, 100% B for 2 min, 100-0% B in 2 min, 0% B for 1 min; flow 1.0 ml/min at 25° C. SEC-HPLC conditions: Yarra 3 μm SEC-4000 300×7.8 mm; A—100 mM Na₂HPO₄, 0.025% NaN₃ pH 6.8; 100% A for 50 min; flow 1.2 ml/min at 25° C.; RP-HPLC conditions: RNASept Prep C18 50×7.8 mm 2 μm; A—100 mM HAA, 10% MeCN, pH 7.0; B—100 mM HAA, 75% MeCN, pH 7.0; 20-70% B in 25 min; flow: 1.0 ml/min at 60° C.

FIG. 10 shows the purification of circRNA using reversed-phase high-pressure liquid chromatography. Products of chemical circularization of pre-circRNA4 were chromatographed under denaturing conditions (60° C., hexylammonium acetate buffer and acetonitrile mixture as a mobile phase). The UV-absorbing eluate was collected and separated into fractions that corresponded to different peaks of absorbance. Next, the fractions were concentrated and analyzed by PAGE, along with EP5-GLuc before and after circularization. NR—precursor (pre-circRNA4), Cr—crude products of circularization, F1-F3—concentrated HPLC fractions, M—linear RNA length marker. HMW refers to linear RNA of length ranging from 3000 nt to 6000 nt. PAGE conditions: 6% AA, AA:MBAA=38:1, 7 M Urea, 1×TBE. Purified circRNA is in fraction 2.

FIG. 11 shows the influence of mobile phase composition on RP-HPLC of circRNA. A mixture of linear (pre-circRNA14) and circular (circRNA14) RNAs was resolved with RP-HPLC. Each chromatography was performed in presence of different ion-pairing agent: A) hexylammonium acetate; B) triethylammonium acetate; C) 2-ethylhexylammonium acetate; D) dibutylammonium acetate; E) N,N-dimethylhexylammonium acetate; F) N,N-diisopropylammonium acetate.

FIG. 12 shows a PAGE analysis of selected circRNA precursors and circRNA after HPLC and RNase R treatment. M-linear RNA length marker. HMW refers to linear RNA of length ranging from 3000 nt to 6000 nt. PAGE conditions: 6% AA, AA:MBAA=38:1, 7 M Urea, 1×TBE.

FIG. 13 shows the translational activity of circRNA analogs in HEK293 cells. Cells were transfected with unmodified, i.e. enzymatically obtained circRNA (circRNA11) or modified, i.e. chemically obtained circRNA (circRNA12 with unnatural linkage, and circRNA13 with a cap structure) encoding Gaussia luciferase, a reporter protein secreted by the cells to the medium. The circRNA precursors (pre-circRNA11-13) were used as references. The cell media were harvested at timepoints (4, 24, 48, 72, 96, 120, 144 and 168 hours post transfection) and used for quantification of the reporter protein levels with luciferase bioluminescence assay. The detected luminescence from all timepoints was summarized to give the total luminescence. The data presented in the graph derives from three independent replicates, error bars represent a standard error of the mean (SEM).

FIG. 14 shows the translational activity of circRNA analogs in HEK 293 cells (continued). Cells were transfected with unmodified (circRNA11) or modified circRNA (circRNA12, 13) encoding Gaussia luciferase, a reporter protein secreted by the cells to the medium. The circRNA precursors (pre-circRNA11-13) were used as a reference. The cell media were harvested at timepoints (4, 24, 48, 72, 96, 120, 144 and 168 hours post transfection) and used for quantification of the reporter protein levels with luciferase bioluminescence assay. The data presented in the graph derives from three independent replicates, error bars represent a standard error of the mean (SEM).

FIG. 15 shows the translational activity of circRNA analog in vitro compared to its linear precursor. HEK 293T, Hep G2, A549, HeLa were transfected with modified circRNA (circRNA12) or its linear precursor (pre-circRNA12) encoding Gaussia luciferase, a reporter protein secreted by the cells to the medium. Mock transfection (water instead of RNA) was used as a reference. Cell media were harvested at timepoints (4, 24, 48, 72, 96, 120, 144 and 168 hours post transfection) and used for quantification of the reporter protein levels with luciferase bioluminescence assay. The detected luminescence from all timepoints was summarized to give the total luminescence, that was subsequently normalizes to the average luminescence obtained for linRNA23. The data presented in the graph derives from three independent repetitions, error bars represent a standard error of the mean (SEM).

### DETAILED DESCRIPTION

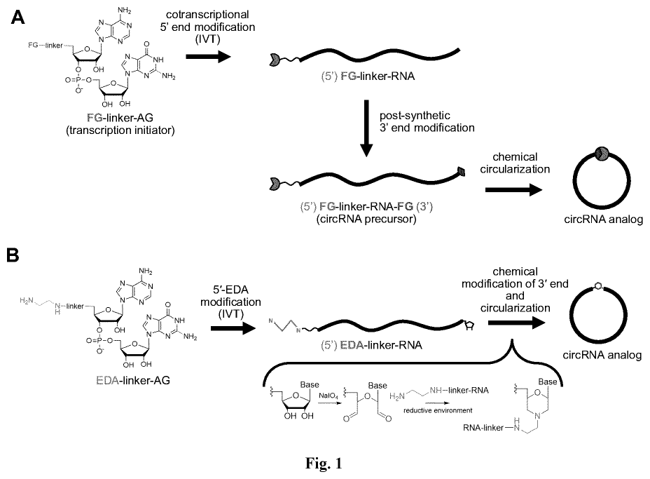

The present invention relates to circular RNA (circRNA) analogs obtained by circularization of a circRNA precursor containing two reactive functional groups, one at the 5′-end and a second at the 3′ end. [FIG. 1A] The reactive functional group at the 5′-end is incorporated into RNA during enzymatic synthesis (i.e. in vitro transcription reaction). The 3′-end reactive functional group is incorporated into RNA post-synthetically, by either enzymatic reaction or, preferably, by chemical reaction. The 5′ and 3′ reactive groups are reactive towards each other either spontaneously or in the presence of a specific trigger, such as the addition of a chemical reagent, addition of a catalysts, a change of temperature, presence of light, or a change in the pH of the solution, etc. Representative pairs of 5′ and 3′ reactive groups reactive towards each other include, but are not limited to: EDA and dialdehyde, hydrazide and dialdehyde, azide and alkyne, azide and cyclooctyne derivative, amino and carboxylic group, amino and fluorosulfonyl group, azide and phosphine, etc.

A representative example of a functional group at the 5′-end is the EDA motif (EDA-linker-RNA). [FIG. 1B] The precursor is a modified nucleic acid that can be prepared by in vitro transcription. The 5′-EDA motif can be readily installed during the in vitro transcription (IVT) reaction if the transcription is carried out in the presence of an EDA-modified transcription initiator [see Table 1 for examples].

This is beneficial, as unlike other methods of chemical circularization, the precursor's length is not limited by the efficiency of RNA solid-phase synthesis.

The 5′-EDA motif and the nucleic acid chain of the EDA-linker-RNA are joined by a linker. The linker itself may be a linear or branched combination of simple chemical moieties (such as carbohydrate or PEG linkers), or it may contain more complex elements, such as detectable labels. The circRNA precursor can bear additional modifications, such as detectable labels, as long as they do not interfere with the circularization reaction.

This is beneficial, as in contrast to enzymatic circularization, the user is not restricted by the activity and substrate-specificity of a particular enzyme.

The chemical circularization reaction is a two-step process. First, the circRNA precursor is selectively oxidized with periodate at the 3′ end to introduce a reactive functional group (a dialdehyde) at the 3′ end of RNA. This occurs via ring-opening oxidation and the formation of a reactive acyclic dialdehyde derivative of the terminal 3′-end ribose. During the second step, the 3′-end dialdehyde derivative spontaneously reacts with the 5′ EDA motif. This step requires reductive environment for optimal performance. If a reductive environment is ensured during this reaction, the reduction of the formed intermediate occurs, making the whole process irreversible. Except for the presence of the 5′ EDA motif (5′ reactive group) and the 3′ dialdehyde motif (3′ reactive group), chemical circularization has no requirements concerning chemical structure or the sequence of the circRNA precursor.

This is beneficial, as in contrast to circularization techniques based on activity of catalytic nucleic acids (PIE and Tornado systems), the invented chemical circularization does not require sequence engineering and construct cloning for preparation of the circRNA precursor.

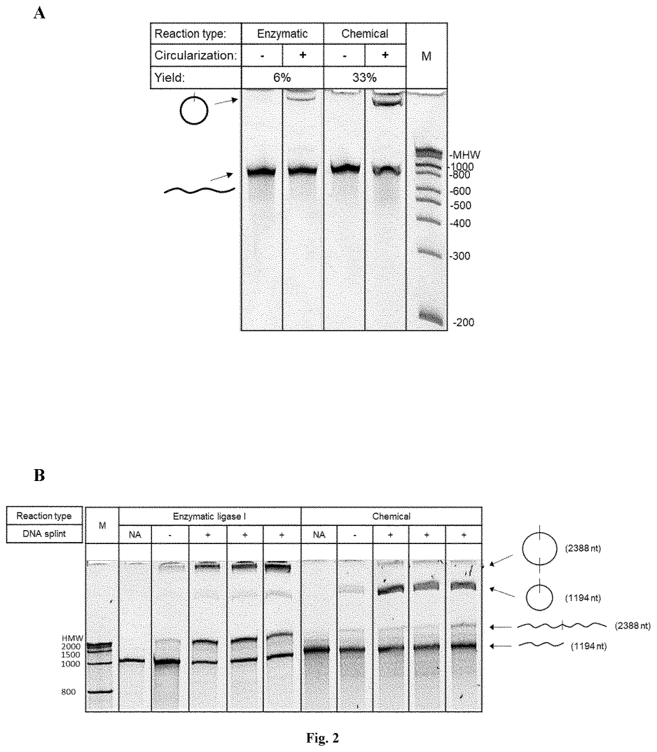

circRNA precursors containing EDA motif were prepared by means of IVT and subsequently circularized [Table 1]. As a comparison, precursors bearing a 5′ monophosphate moiety and an identical sequence were subjected to enzymatic circularization catalysed by T4 RNA ligase I or T4 RNA ligase II. Unexpectedly, the yield of chemical circularization, in comparison to enzymatic circularization, is less dependent on the precursor sequence and is often more efficient [FIG. 2A]. In some of the investigated enzymatic circularization processes, the predominant outcome involves the generation of linear and circular dimers, while the chemical circularization predominantly results in the formation of the desired circular RNA product [FIG. 2B].

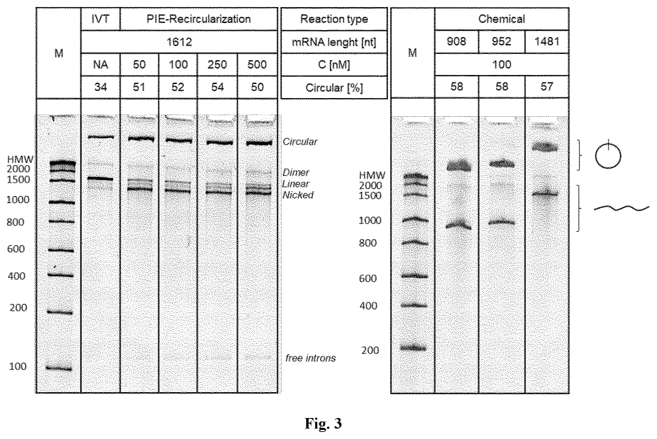

The method for chemical circularization of mRNAs as disclosed herein is applicable for circularization of long RNA molecules. Crude products of circularization reaction are a non-complex mixture composed of the desired circRNA product and unreacted linear pre-circRNA. The yield of circularization reaction is high and independent of pre-circRNA length. The circularization procedure requires no additional steps, in contrast to PIE methodology that often requires a recircularization step. The above-mentioned recircularization step, due to prolonged incubation of RNA in presence of magnesium salts, can lead to degradation (nicking) of the desired circRNA product, as well as the formation of a complex mixture of by-products, which can interfere with the subsequent purification steps [FIG. 3]. The circular topology of the circRNA analogs obtained by chemical circularization can be verified with exonucleolytic digestion assays, endonucleolytic assays, as well as electrophoretic mobility assays.

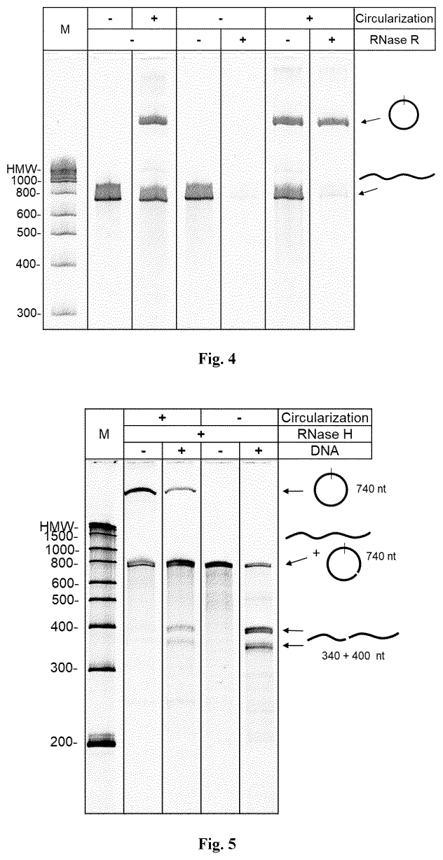

Exonucleolytic digestion assays allow one to distinguish linear RNAs from circular RNAs, as exonucleases can hydrolyse only RNA molecules that contain free 5′ or 3′ end. On the other hand, circular RNAs are resistant to exonucleolytic hydrolysis as they lack free 5′ and 3′ ends. Rnase R is a processive 3′ exonuclease, that is most commonly used for such assay.³ The products of chemical circularization were treated with Rnase R [FIG. 4]. In contrary to the precursors, the circRNA analogs were resistant to Rnase R hydrolysis.

Endonucleolytic assays are based on a phenomenon of nicking and linearization of circRNA. Site-selective and sequence-dependent cleavage of a circular RNA molecule leads to a single linear RNA molecule of equal size. On the other hand, cleavage of a linear RNA molecule leads to two linear RNA molecules of smaller sizes. Such assay can be performed with Rnase H, an endonuclease, that nicks RNA in RNA-DNA duplexes.²⁵ Rnase H and targeting DNA oligonucleotides were used for nicking of the products of chemical circularization [FIG. 5]. The assay confirmed that, in contrast to the precursors, nicking of the circRNA analogs leads to their linearization.

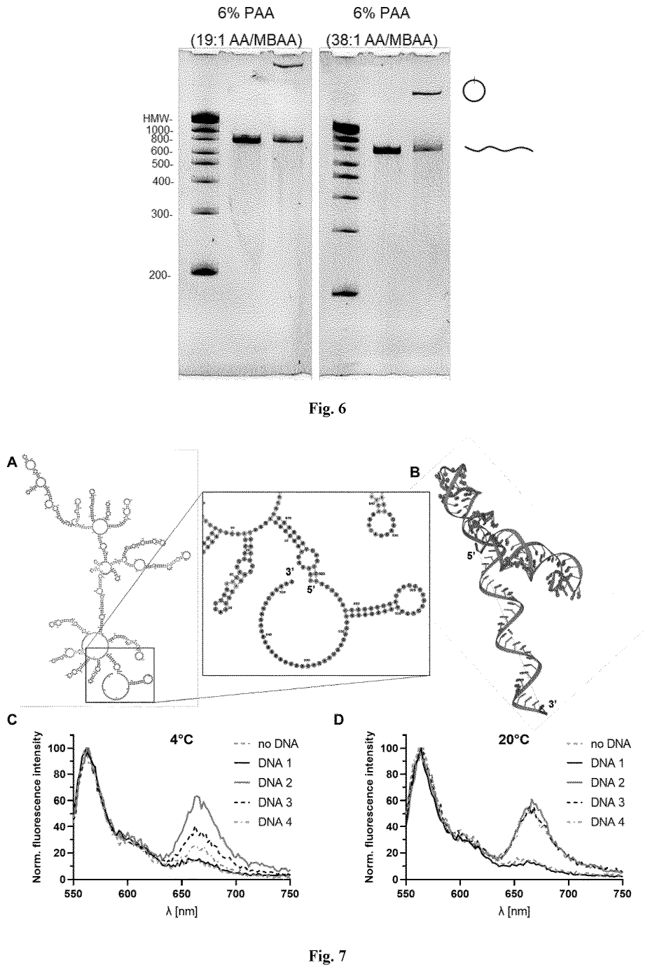

Electrophoretic mobility of nucleic acids is affected by their size, charge, conformation, and topology.^{26, 27} Thanks to that, circRNA and their precursors can be separated during polyacrylamide gel electrophoresis in denaturing conditions [FIG. 6]. The crosslinking degree of the polyacrylamide gel matrix is a crucial factor for the separation of RNA based on their topology. Electrophoretic separation of linear and circular RNA using a cross-linked polyacrylamide gel is significantly better than with regular or commercially available agarose gels.²⁷

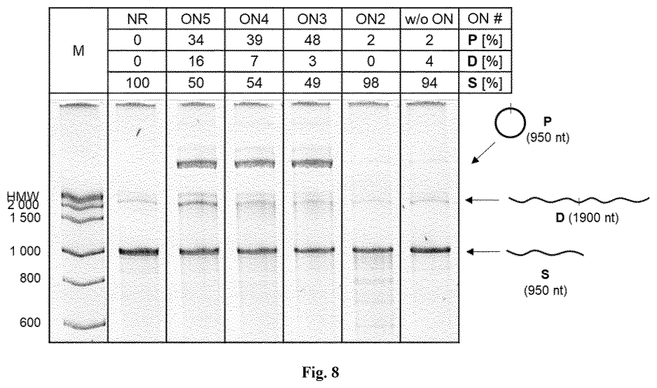

The circularization efficiency is affected by the sequence of the circRNA precursor. [Tab. 2] In some cases, such as sequences containing a 3′ polyA sequence, the circularization yield can be increased by the addition of a complementary nucleic acid oligomer. [Tab. 3] During the reaction, the oligomer and precursor hybridize and affect the conformation of the precursor. Using an oligomer with an optimal design, the conformational change of the precursor decreases the distance between the 5′ and 3′ ends [FIG. 7]. In consequence, the optimal distance and orientation of the 5′ and 3′ ends increase the circularization yield [FIG. 8].

The present invention also relates to methods of preparation and isolation of circRNA analogs. This includes development of chromatographic methods of linear and circular RNA resolution.

Previously reported methods of chromatography of circularization products were based on size-exclusion chromatography in an HPLC setup (SEC-HPLC).^{11, 12, 14} This method allows one to separate sample components based on hydrodynamic radius and is considered to provide one of the mildest chromatographic conditions. SEC-HPLC was used to resolve the products of PIE-based circularization.^{11, 12, 14} In these cases, the precursor and circRNA significantly differ in molecular size between each other, as well as form side products (such as free introns). In case of synthetic polymers (such as polystyrene or poli(ethyneneoxide)) the hydrodynamic radius (Stokes radius) is decreased upon circularization.²⁸ This property was used to separate linear and cyclic polymers using gel permeation chromatography (GPC). However, the decrease in the Stokes radius of an RNA molecule caused by its circularization has not been documented. One could speculate, that intramolecular interactions, that contribute to the existence of a landscape of RNA conformers, may diminish the influence of topological change on the differentiation of Stokes radii of circular and linear RNA.²⁹ This could also explain why SEC-HPLC under mild conditions leads to the coelution of intact and nicked circRNA.^{12, 14}

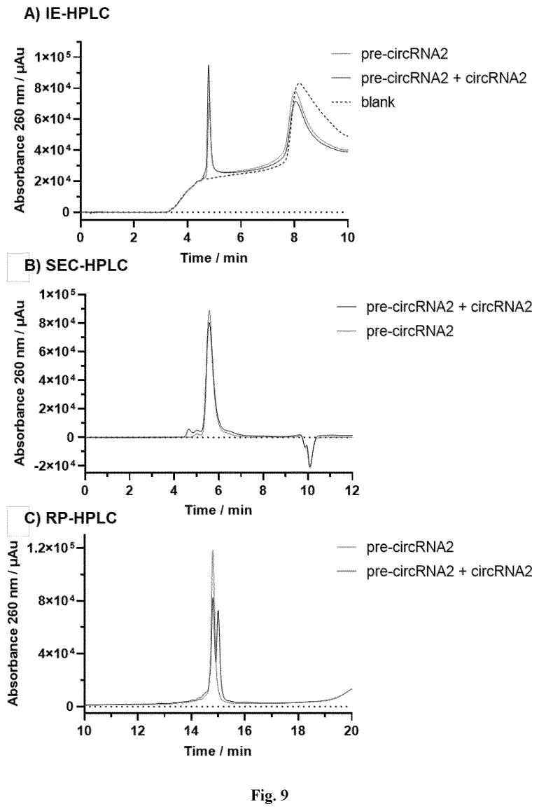

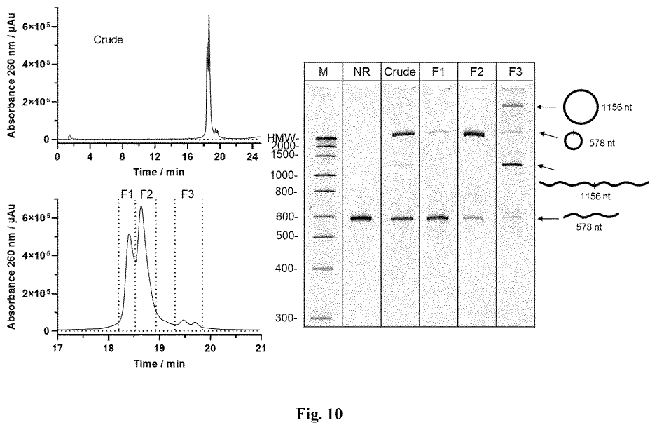

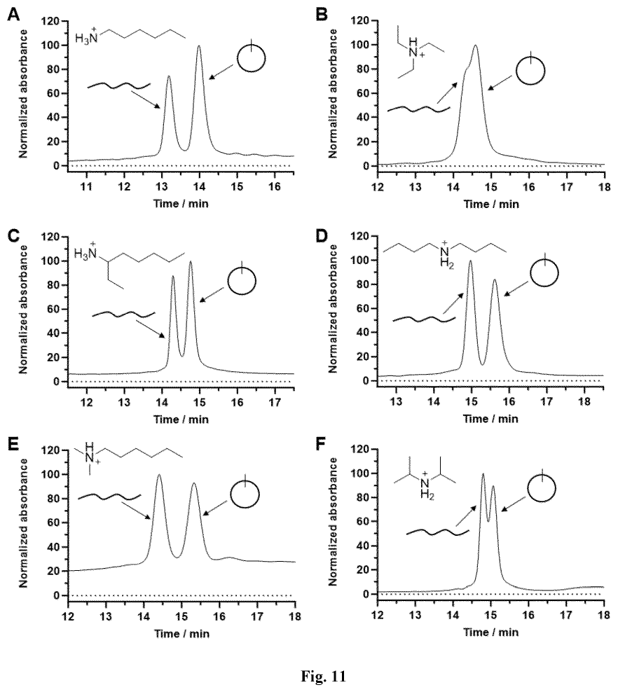

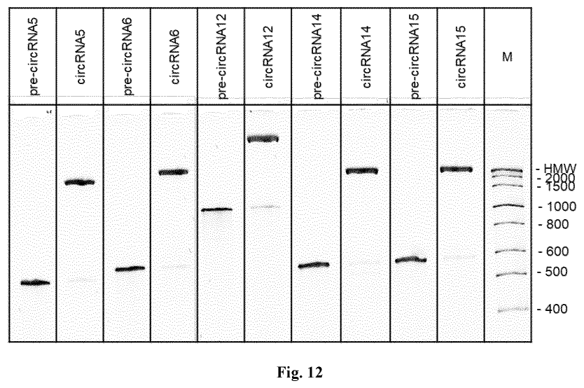

During the development of the invention, we found that both size-exclusion chromatography (SEC-HPLC) and ion-exchange HPLC (IE-HPLC) are insufficiently effective for separation of linear and circular RNA of the same molar mass [FIG. 9]. Reversed-phase HPLC (RP-HPLC) in ion-pair mode was found to be the most suitable method for the separation of circularization products [FIG. 9, 10]. Chromatography was performed using polistyren-divinylbenzen (PSDVB) resins, elevated temperature (50-80° C.), and a mixture of an aqueous solution of ion pair reagent with acetonitrile as a mobile phase. The separation of linear and circular RNA is affected by composition of the mobile phase, namely the chosen ion pair reagent. The most effective resolution was obtained with 100 mM n-hexylammonium acetate (HAA), with a linear gradient of acetonitrile (45-58.5% in 25 min, flowrate 1.00 ml/min at 60° C.) [FIG. 11].

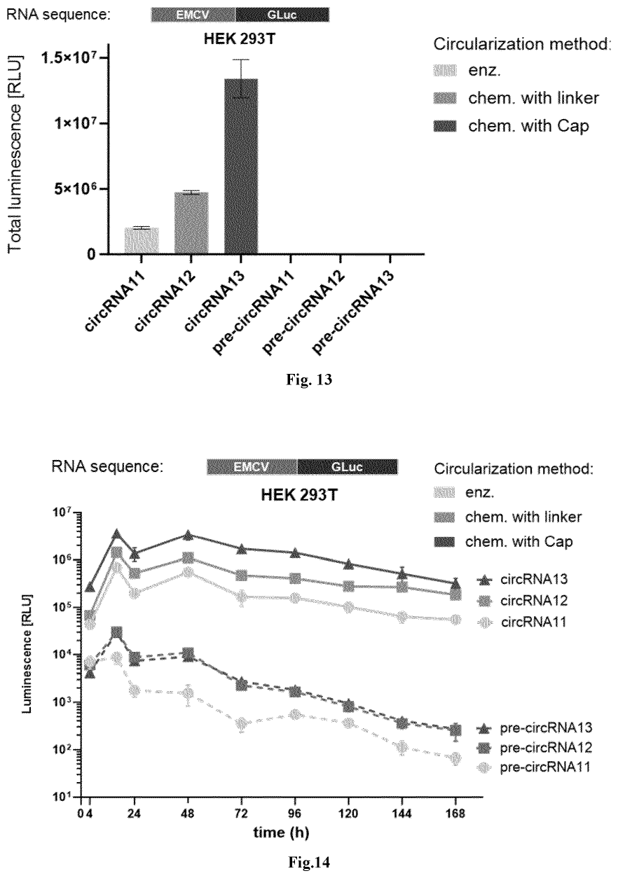

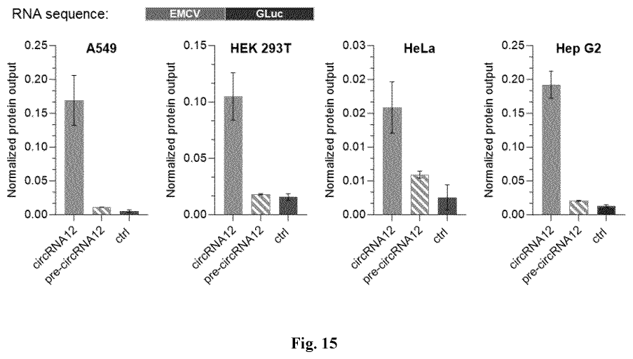

The present invention also relates to therapeutic application of circRNA analogs. To demonstrate the applicability of protein-encoding circRNA analogs in gene delivery, circRNA analogs encoding the reporter protein, Gaussia luciferase were synthesized and expressed in cell lines HEK 293T, A549, HeLa and Hep G2. The levels of the reporter protein were higher in case of expression of chemically modified circRNA analogs (circRNA12, 13) than unmodified circRNA (circRNA11) [FIG. 13]. Both modified and unmodified circRNAs showed increased duration of protein expression in comparison to their linear precursors (pre-circRNA11-12) [FIG. 14]. The chemically circularized mRNAs had higher translation efficiency compared to their liner precursors [FIG. 15]. The result shows that chemical modification within the circRNA analogs is beneficial in the context of therapeutic protein expression.

### TABLES AND THEIR BRIEF DESCRIPTIONS

TABLE 1
List of DNA templates (linearized plasmids) and corresponding
RNA sequences with description and properties.

| | | | | | | | RNA | |
|---|---|---|---|---|---|---|---|---|
| # | | # RNA | RNA sequence elements | | | | length | RNA E₂₆₀ |
| Plasmid | Restrictase | sequence | 5′ UTR | CDS | 3′ UTR | 3′ tail | [nt] | [μM⁻¹cm⁻¹] |
| PL1 | BamHI | RNA1 | — | eGFP | — | — | 740 | 8.93 |
| PL2 | BamHI | RNA2 | — | Gluc | — | — | 578 | 6.99 |
| PL3 | BamHI | RNA3 | HBB | | — | — | 626 | 7.58 |
| | PmeI | RNA4 | | | HBB × 2 | — | 918 | 11.0 |
| | AarI | RNA5 | | | HBB × 2 | A30 | 952 | 11.5 |
| PL4 | BamHI | RNA6 | EMCV | | — | — | 1152 | 13.9 |
| PL5 | | RNA7 | RSV | | — | — | 598 | 7.25 |
| PL6 | | RNA8 | OligoIRES | | — | — | 711 | 9.35 |
| PL7 | Esp3I | RNA9 | EMCV | | HBB × 2 | A30 | 1446 | 17.86 |
| PL8 | AarI | RNA10 | Kozak | | HBB × 2 | A30 | 908 | 10.99 |
| PL9 | Esp3I | RNA11 | EMCV | eGFP | — | pAC | 1492 | 13.51 |
| PL10 | Eco52I | RNA12 | EMCV | Gluc | — | pAC | 1612 | 19.61 |

Column description: # plasmid—designation of a plasmid; Restrictase—name of the enzyme used for linearization of the plasmid; # RNA sequence—designation of a RNA sequence encoded in the DNA template for IVT; RNA sequence elements—5′ and 3′ untranslated regions (5′ UTR and 3′ UTR), protein-coding sequence (CDS), sequence downstream of the 3′ UTR (3′ tail); RNA length—total length of the transcript; RNA E260—molar extinction coefficient calculated for particular RNA sequence.³⁰
HBB: human betaglobin UTR (5′ or 3′) sequence, EMCV: Encephalomyocarditis virus IRES, RSV: respiratory syncytial virus IRES, OligoIRES: synthetic IRES, eGFP: enhanced green fluorescent protein sequence, Gluc: Gaussia-Dura luciferase sequence, A30: 30 nucleotide-long poliadenine sequence.

TABLE 2
List of circRNA and yields of their chemical or enzymatic circularization.
Column description: # circRNA - designation of a circRNA and corresponding
pre-circRNA; # RNA sequence - designation of an RNA sequence encoded
in the DNA template for IVT; IVT initiator - compound used as transcription
initiator in IVT reaction; Circularization type - type of reaction (chemical
or enzymatic catalyzed by T4RNA ligase I or T4 RNA ligase II) used for RNA
circularization; DNA oligonucleotide - information on whether a complementary
DNA oligonucleotide was used during particular circularization reaction;
Circularization yield - fraction of the circRNA precursor converted to particular
circRNA product during circularization reaction, determined by PAGE of crude
circularization products and densitometric analysis.

| | # RNA | IVT | Circularization | DNA | Circularization |
|---|---|---|---|---|---|
| # circRNA | sequence | initiator | type | oligonucleotide | yield [%] |
| circRNA1 | RNA1 | GMP | Enz. (T4 Lig. 1) | w/o ON | 5-10 |
| circRNA2 | | EDA- | Chem. | | 30-45 |
| | | PEG₅-AG | | | |
| circRNA3 | RNA2 | GMP | Enz. (T4 Lig. 1) | | 36 |
| circRNA4 | | EDA- | Chem. | | 60 |
| | | PEG₅-AG | | | |
| circRNA5 | | EDA- | | | 53 |
| | | PEG₅- | | | |
| | | Cap1 | | | |
| circRNA6 | RNA3 | EDA- | Chem. | | 46 |
| | | PEG₅- | | | |
| | | Cap1 | | | |
| circRNA7 | RNA4 | GMP | Enz. (T4 Lig. 1) | w/o ON | 13 |
| | | | Enz. (T4 Lig. 2) | w/o ON | 31 |
| | | | | ON1 | 1-45 |
| circRNA8 | | EDA- | Chem. | w/o ON | 10 |
| | | PEG₅- | | ON1 | 50 |
| | | Cap1 | | | |
| circRNA9 | RNA5 | GMP | Enz. (T4 Lig. 1) | w/o ON | 9 |
| | | | Enz. (T4 Lig. 2) | w/o ON | 32 |
| | | | | ON4 | 61 |
| circRNA10 | | EDA- | Chem | w/o ON | 2 |
| | | PEG₅- | | ON4 | 40 |
| | | Cap1 | | | |
| circRNA11 | RNA6 | GMP | Enz. (T4 Lig. 1) | w/o ON | 10 |
| | | | Enz. (T4 Lig. 2) | ON6 | 46 |
| circRNA12 | | EDA- | Chem | w/o ON | 44 |
| | | PEG₅-AG | | ON6 | 44 |
| circRNA13 | | EDA- | | ON6 | 41 |
| | | PEG₅- | | | |
| | | Cap1 | | | |
| circRNA14 | RNA7 | GMP | Enz. (T4 Lig. 1) | w/o ON | 40 |
| | | | Enz. (T4 Lig. 2) | | 84 |
| circRNA15 | | EDA- | Chem | | 52 |
| | | PEG₅-AG | | | |
| circRNA16 | RNA8 | GMP | Enz. (T4 Lig. 1) | | 5-10 |
| circRNA17 | | EDA- | Chem | | 53 |
| | | PEG₅-AG | | | |
| circRNA18 | RNA9 | EDA- | Chem | ON7 | 57 |
| | | PEG₅- | | | |
| | | Cap1 | | | |
| circRNA19 | RNA10 | EDA- | Chem | ON8 | 58 |
| | | PEG₅- | | | |
| | | Cap1 | | | |
| circRNA20 | RNA11 | GMP | Enz. (T4 Lig. 1) | ON9 | 1-3 |
| circRNA21 | | EDA- | Chem | | 37-51 |
| | | PEG₅-AG | | | |
| circRNA22 | RNA12 | NA | PIE | NA | 34-54 |
| linRNA23 | RNA05 | Cap1 | NA | NA | NA |

TABLE 3
List of complementary DNA oligonucleotides used for RNA circularization and FRET
experiments. Column descriptions: #ON-designation of a DNA oligonucleotide sequence;
# RNA sequence-designation of an RNA sequence; ON sequence-sequence of the designated
oligonucleotide.

| # | # RNA | |
|---|---|---|
| ON | sequence | ON sequence |
| ON1 | RNA4 | AGCAAATGTCTAGATTATCCCTAACATTTAAATGCAATGAAAA |
| ON2 | RNA5 | TCCCTTTTTT |
| ON3 | | TCTAGATTATCCCTTTTTTTTTTTTTTT |
| ON4 | | GAAGCAAATGTCTAGATTATCCCTTTTTTTTTTTTTTTTTTTTTTTTT |
| ON5 | | GAAGCAAATGTCTAGATTATCCCTTTTTTTTTTTTTTTTTTTTTTTTTTTTTTTGTTTAAACATTTA |
| ON6 | RNA6 | AGGGGGGGAGGGAGAGGGGTACCCCCTATCCTTAGTCACCACCGGCCCCCT |
| ON7 | RNA9 | GGGAGAGGGGTACCCCCTTTTTTTTTTTTTTTTTTTTT |
| ON8 | RNA10 | CTTTGACTCCCATGGTGGCGTACCCCCTTTTTTTTTTTTTTTTTTTTT |
| ON9 | RNA11 | TGTTTTGTTTTACCCCTTTGGTTTTTTTTTT |

TABLE 4
List of plasmids and inserted sequences, used for preparation of IVT templates.
Underlined is the coding sequence from the T7 RNA polymerase transcription start site
to the site of linearization. The recognition sequence for BamHI (GGATCC), PmeI
(GTTTAAAC) and AarI (AAAAAAAAAGCAGGTG), Esp3I (NNCGTCTC) and Eco52I (CGGCCG) are in
bold with the cut site between nucleotides in italic.

| | |
|---|---|
| PL1 | GCCGAATTATATTATTTTTGCCAAATAATTTTTAACAAAAGCTCTGAAGTCTTCTTCATTTAAATTC |
| | TTAGATGATACTTCATCTGGAAAATTGTCCCAATTAGTAGCATCACGCTGTGTAATACGACTCACT |
| | ATAGGGGGTACGCCACCATGGTGAGCAAGGGCGAGGAGCTGTTCACCGGGGTGGTGCCCATCCTG |
| | GTCGAGCTGGACGGCGACGTAAACGGCCACAAGTTCAGCGTGTCCGGCGAGGGCGAGGGCGATGC |
| | CACCTACGGCAAGCTGACCCTGAAGTTCATCTGCACCACCGGCAAGCTGCCCGTGCCCTGGCCCAC |
| | CCTCGTGACCACCCTGACCTACGGCGTGCAGTGCTTCAGCCGCTACCCCGACCACATGAAGCAGCA |
| | CGACTTCTTCAAGTCCGCCATGCCCGAAGGCTACGTCCAGGAGCGCACCATCTTCTTCAAGGACGA |
| | CGGCAACTACAAGACCCGCGCCGAGGTGAAGTTCGAGGGCGACACCCTGGTGAACCGCATCGAGC |
| | TGAAGGGCATCGACTTCAAGGAGGACGGCAACATCCTGGGGCACAAGCTGGAGTACAACTACAAC |
| | AGCCACAACGTCTATATCATGGCCGACAAGCAGAAGAACGGCATCAAGGTGAACTTCAAGATCCG |
| | CCACAACATCGAGGACGGCAGCGTGCAGCTCGCCGACCACTACCAGCAGAACACCCCCATCGGCG |
| | ACGGCCCCGTGCTGCTGCCCGACAACCACTACCTGAGCACCCAGTCCGCCCTGAGCAAAGACCCC |
| | AACGAGAAGCGCGATCACATGGTCCTGCTGGAGTTCGTGACCGCCGCCGGGATCACTCTCGGCAT |
| | GGACGAGCTGTACAAGTAAGGATCCTAGGGCCGCAGCTCGCTTTCTTGCTGTCCAATTTCTATTAA |
| | AGGTTCCTTTGTTCCCTAAGTCCAACTACTAAACTGGGGGATATTATGAAGGGCCTTGAGCATTTG |
| | GATTCTGCCTAATAAAAAACATTTATTTTCATTGCGTTTAGCTCGCTTTCTTGCTGTCCAATTTCTAT |
| | TAAAGGTTCCTTTGTTCCCTAAGTCCAACTACTAAACTGGGGGATATTATGAAGGGCCTTGAGCAT |
| | TTGGATTCTGCCTAATAAAAAACATTTATTTTCATTGCATTTAAATGTTTAAACAAAAAAAAAAAA |
| | AAAAAAAAAAAAAAAAAAAAAAAAAAAAAAAAAAAAAAAAAAAAAAAAAAAAAAAAAAAAAA |
| | AAAAAAAAAAAAAAAAAAAAAAAAAAAAAAAAAAAAAAAAAAAAAAAAAAAAAAGCAGGTGG |
| | GAGGGTACGTTCGTGATAAACAAAC |
| PL2 | GCCCCTGCAGCCGAATTATATTATTTTTGCCAAATAATTTTTAACAAAAGCTCTGAAGTCTTCTTCA |
| | TTTAAATTCTTAGATGATACTTCATCTGGAAAATTGTCCCAATTAGTAGCATCACGCTGTGTAATAC |
| | GACTCACTATAGGGGGTACGCCACCATGGGAGTCAAAGTTCTGTTTGCCCTGATCTGCATCGCTGT |
| | GGCCGAGGCCAAGCCCACCGAGAACAACGAAGACTTCAACATCGTGGCCGTGGCCAGCAACTTCG |
| | CGACCACGGATCTCGATGCTGACCGCGGGAAGTTGCCCGGCAAGAAGCTGCCGCTGGAGGTGCTC |
| | AAAGAGTTGGAAGCCAATGCCCGGAAAGCTGGCTGCACCAGGGGCTGTCTGATCTGCCTGTCCCA |
| | CATCAAGTGCACGCCCAAGATGAAGAAGTTCATCCCAGGACGCTGCCACACCTACGAAGGCGACA |
| | AAGAGTCCGCACAGGGCGGCATAGGCGAGGCGATCGTCGACATTCCTGAGATTCCTGGGTTCAAG |
| | GACTTGGAGCCCTTGGAGCAGTTCATCGCACAGGTCGATCTGTGTGTGGACTGCACAACTGGCTGC |
| | CTCAAAGGGCTTGCCAACGTGCAGTGTTCTGACCTGCTCAAGAAGTGGCTGCCGCAACGCTGTGCG |
| | ACCTTTGCCAGCAAGATCCAGGGCCAGGTGGACAAGATCAAGGGGGCCGGTGGTGACTAAGGAT |
| | CCTAGGGCCGCAGCTCGCTTTCTTGCTGTCCAATTTCTATTAAAGGTTCCTTTGTTCCCTAAGTCCA |
| | ACTACTAAACTGGGGGATATTATGAAGGGCCTTGAGCATTTGGATTCTGCCTAATAAAAAACATTT |
| | ATTTTCATTGCGTTTAGCTCGCTTTCTTGCTGTCCAATTTCTATTAAAGGTTCCTTTGTTCCCTAAGT |
| | CCAACTACTAAACTGGGGGATATTATGAAGGGCCTTGAGCATTTGGATTCTGCCTAATAAAAAACA |
| | TTTATTTTCATTGCATTTAAATGTTTAAACAAAAAAAAAAAAAAAAAAAAAAAAAAAAAAAAAAA |
| | AAAAAAAAAAAAAAAAAAAAAAAAAAAAAAAAAAAAAAAAAAAAAAAAAAAAAAAAAAAAAA |
| | AAAAAAAAAAAAAAAAAAAAAAAAAAAAAAAGCAGGTGGGAGGGTACGTTCGTGATAAACAAA |
| | C |
| PL3 | CTGCAGCCGAATTATATTATTTTTGCCAAATAATTTTTAACAAAAGCTCTGAAGTCTTCTTCATTTA |
| | AATTCTTAGATGATACTTCATCTGGAAAATTGTCCCAATTAGTAGCATGCCACGCTGTGTAATACG |
| | ACTCACTATAGGGATAATCTAGACATTTGCTTCTGACACAACTGTGTTCACTAGCAACCTCAAACA |
| | GACACCATGGGAGTCAAAGTTCTGTTTGCCCTGATCTGCATCGCTGTGGCCGAGGCCAAGCCCACC |
| | GAGAACAACGAAGACTTCAACATCGTGGCCGTGGCCAGCAACTTCGCGACCACGGATCTCGATGC |
| | TGACCGCGGGAAGTTGCCCGGCAAGAAGCTGCCGCTGGAGGTGCTCAAAGAGTTGGAAGCCAATG |
| | CCCGGAAAGCTGGCTGCACCAGGGGCTGTCTGATCTGCCTGTCCCACATCAAGTGCACGCCCAAGA |
| | TGAAGAAGTTCATCCCAGGACGCTGCCACACCTACGAAGGCGACAAAGAGTCCGCACAGGGCGGC |
| | ATAGGCGAGGCGATCGTCGACATTCCTGAGATTCCTGGGTTCAAGGACTTGGAGCCCTTGGAGCAG |
| | TTCATCGCACAGGTCGATCTGTGTGTGGACTGCACAACTGGCTGCCTCAAAGGGCTTGCCAACGTG |
| | CAGTGTTCTGACCTGCTCAAGAAGTGGCTGCCGCAACGCTGTGCGACCTTTGCCAGCAAGATCCAG |
| | GGCCAGGTGGACAAGATCAAGGGGGCCGGTGGTGACTAAGGATCCTAGGGCCGCAGCTCGCTTTC |
| | TTGCTGTCCAATTTCTATTAAAGGTTCCTTTGTTCCCTAAGTCCAACTACTAAACTGGGGGATATTA |
| | TGAAGGGCCTTGAGCATTTGGATTCTGCCTAATAAAAAACATTTATTTTCATTGCGTTTAGCTCGCT |
| | TTCTTGCTGTCCAATTTCTATTAAAGGTTCCTTTGTTCCCTAAGTCCAACTACTAAACTGGGGGATA |
| | TTATGAAGGGCCTTGAGCATTTGGATTCTGCCTAATAAAAAACATTTATTTTCATTGCATTTAAATG |
| | TTTAAACAAAAAAAAAAAAAAAAAAAAAAAAAAAAAAAAAAGCAGGTGGGAGGGTACGTTCGT |
| | GATAAACAAACGGCCGC |
| PL4 | CCTGCAGCCGAATTATATTATTTTTGCCAAATAATTTTTAACAAAAGCTCTGAAGTCTTCTTCATTT |
| | AAATTCTTAGATGATACTTCATCTGGAAAATTGTCCCAATTAGTAGCATGCCACGCTGTGTAATAC |
| | GACTCACTATAGGGGGTACCCCTCTCCCTCCCCCCCTAACGTTACTGGCCGAAGCCGCTTGGAATA |
| | AGGCCGGTGTGCGTTTGTCTATATGTTATTTTCCACCATATTGCCGTCTTTTGGCAATGTGAGGGCC |
| | CGGAAACCTGGCCCTGTCTTCTTGACGAGCATTCCTAGGGGTCTTTCCCCTCTCGCCAAAGGAATG |
| | CAAGGTCTGTTGAATGTCGTGAAGGAAGCAGTTCCTCTGGAAGCTTCTTGAAGACAAACAACGTCT |
| | GTAGCGACCCTTTGCAGGCAGCGGAACCCCCCACCTGGCGACAGGTGCCTCTGCGGCCAAAAGCC |
| | ACGTGTATAAGATACACCTGCAAAGGCGGCACAACCCCAGTGCCACGTTGTGAGTTGGATAGTTGT |
| | GGAAAGAGTCAAATGGCTCTCCTCAAGCGTATTCAACAAGGGGCTGAAGGATGCCCAGAAGGTAC |
| | CCCATTGTATGGGATCTGATCTGGGGCCTCGGTGCACATGCTTTACATGTGTTTAGTCGAGGTTAA |
| | AAAACGTCTAGGCCCCCCGAACCACGGGGACGTGGTTTTCCTTTGAAAAACACGATGATAATATG |
| | GCCACACATATGGGAGTCAAAGTTCTGTTTGCCCTGATCTGCATCGCTGTGGCCGAGGCCAAGCCC |
| | ACCGAGAACAACGAAGACTTCAACATCGTGGCCGTGGCCAGCAACTTCGCGACCACGGATCTCGA |
| | TGCTGACCGCGGGAAGTTGCCCGGCAAGAAGCTGCCGCTGGAGGTGCTCAAAGAGTTGGAAGCCA |
| | ATGCCCGGAAAGCTGGCTGCACCAGGGGCTGTCTGATCTGCCTGTCCCACATCAAGTGCACGCCCA |
| | AGATGAAGAAGTTCATCCCAGGACGCTGCCACACCTACGAAGGCGACAAAGAGTCCGCACAGGGC |
| | GGCATAGGCGAGGCGATCGTCGACATTCCTGAGATTCCTGGGTTCAAGGACTTGGAGCCCTTGGAG |
| | CAGTTCATCGCACAGGTCGATCTGTGTGTGGACTGCACAACTGGCTGCCTCAAAGGGCTTGCCAAC |
| | GTGCAGTGTTCTGACCTGCTCAAGAAGTGGCTGCCGCAACGCTGTGCGACCTTTGCCAGCAAGATC |
| | CAGGGCCAGGTGGACAAGATCAAGGGGGCCGGTGGTGACTAAGGATCCTAGGGCCGCAGCTCGC |
| | TTTCTTGCTGTCCAATTTCTATTAAAGGTTCCTTTGTTCCCTAAGTCCAACTACTAAACTGGGGGAT |
| | ATTATGAAGGGCCTTGAGCATTTGGATTCTGCCTAATAAAAAACATTTATTTTCATTGCGTTTAGCT |
| | CGCTTTCTTGCTGTCCAATTTCTATTAAAGGTTCCTTTGTTCCCTAAGTCCAACTACTAAACTGGGG |
| | GATATTATGAAGGGCCTTGAGCATTTGGATTCTGCCTAATAAAAAACATTTATTTTCATTGCATTTA |
| | AATGTTTAAACAAAAAAAAAAAAAAAAAAAAAAAAAAAAAAAAAAGCAGGTGGGAGGGTACGT |
| | TCGTGATAAACAAACGGCCGCT |
| PL5 | CCTGCAGCCGAATTATATTATTTTTGCCAAATAATTTTTAACAAAAGCTCTGAAGTCTTCTTCATTT |
| | AAATTCTTAGATGATACTTCATCTGGAAAATTGTCCCAATTAGTAGCATGCCACGCTGTGTAATAC |
| | GACTCACTATAGGGGGTAATGAGTCCTGGACTGAAACGGACTCATATGGGAGTCAAAGTTCTGTTT |
| | GCCCTGATCTGCATCGCTGTGGCCGAGGCCAAGCCCACCGAGAACAACGAAGACTTCAACATCGT |
| | GGCCGTGGCCAGCAACTTCGCGACCACGGATCTCGATGCTGACCGCGGGAAGTTGCCCGGCAAGA |
| | AGCTGCCGCTGGAGGTGCTCAAAGAGTTGGAAGCCAATGCCCGGAAAGCTGGCTGCACCAGGGGC |
| | TGTCTGATCTGCCTGTCCCACATCAAGTGCACGCCCAAGATGAAGAAGTTCATCCCAGGACGCTGC |
| | CACACCTACGAAGGCGACAAAGAGTCCGCACAGGGCGGCATAGGCGAGGCGATCGTCGACATTCC |
| | TGAGATTCCTGGGTTCAAGGACTTGGAGCCCTTGGAGCAGTTCATCGCACAGGTCGATCTGTGTGT |
| | GGACTGCACAACTGGCTGCCTCAAAGGGCTTGCCAACGTGCAGTGTTCTGACCTGCTCAAGAAGTG |
| | GCTGCCGCAACGCTGTGCGACCTTTGCCAGCAAGATCCAGGGCCAGGTGGACAAGATCAAGGGGG |
| | CCGGTGGTGACTAAGGATCCTAGGGCCGCAGCTCGCTTTCTTGCTGTCCAATTTCTATTAAAGGTT |
| | CCTTTGTTCCCTAAGTCCAACTACTAAACTGGGGGATATTATGAAGGGCCTTGAGCATTTGGATTCT |
| | GCCTAATAAAAAACATTTATTTTCATTGCGTTTAGCTCGCTTTCTTGCTGTCCAATTTCTATTAAAG |
| | GTTCCTTTGTTCCCTAAGTCCAACTACTAAACTGGGGGATATTATGAAGGGCCTTGAGCATTTGGA |
| | TTCTGCCTAATAAAAAACATTTATTTTCATTGCATTTAAATGTTTAAACAAAAAAAAAAAAAAAAA |
| | AAAAAAAAAAAAAAAAAGCAGGTGGGAGGGTACGTTCGTGATAAACAAACGGCCGCT |
| PL6 | GCCCCTGCAGCCGAATTATATTATTTTTGCCAAATAATTTTTAACAAAAGCTCTGAAGTCTTCTTCA |
| | TTTAAATTCTTAGATGATACTTCATCTGGAAAATTGTCCCAATTAGTAGCATGCCACGCTGTGTAAT |
| | ACGACTCACTATAGGGGGTAGGCGCGCCAGTCCTTCTGTAGCGACCCTTTGCAGGCAGCGGAACCC |
| | CCCACCTGGCGACAGGTGCCTCTGCGGCCAAAAGCCACGTGTATAAGATACACCTGCAAAGGCGG |
| | CACAACCCCAGTGCCACGTTGTGAGTTGGATAGTTGTGGAAAGAGTCAAATGGCTCTCCTCAAGCG |
| | TATTCAACAAGCGACTCGGACATATGGGAGTCAAAGTTCTGTTTGCCCTGATCTGCATCGCTGTGG |
| | CCGAGGCCAAGCCCACCGAGAACAACGAAGACTTCAACATCGTGGCCGTGGCCAGCAACTTCGCG |
| | ACCACGGATCTCGATGCTGACCGCGGGAAGTTGCCCGGCAAGAAGCTGCCGCTGGAGGTGCTCAA |
| | AGAGTTGGAAGCCAATGCCCGGAAAGCTGGCTGCACCAGGGGCTGTCTGATCTGCCTGTCCCACAT |
| | CAAGTGCACGCCCAAGATGAAGAAGTTCATCCCAGGACGCTGCCACACCTACGAAGGCGACAAAG |
| | AGTCCGCACAGGGCGGCATAGGCGAGGCGATCGTCGACATTCCTGAGATTCCTGGGTTCAAGGAC |
| | TTGGAGCCCTTGGAGCAGTTCATCGCACAGGTCGATCTGTGTGTGGACTGCACAACTGGCTGCCTC |
| | AAAGGGCTTGCCAACGTGCAGTGTTCTGACCTGCTCAAGAAGTGGCTGCCGCAACGCTGTGCGACC |
| | TTTGCCAGCAAGATCCAGGGCCAGGTGGACAAGATCAAGGGGGCCGGTGGTGACTAAGGATCCTA |
| | GGGCCGCAGCTCGCTTTCTTGCTGTCCAATTTCTATTAAAGGTTCCTTTGTTCCCTAAGTCCAACTA |
| | CTAAACTGGGGGATATTATGAAGGGCCTTGAGCATTTGGATTCTGCCTAATAAAAAACATTTATTT |
| | TCATTGCGTTTAGCTCGCTTTCTTGCTGTCCAATTTCTATTAAAGGTTCCTTTGTTCCCTAAGTCCAA |
| | CTACTAAACTGGGGGATATTATGAAGGGCCTTGAGCATTTGGATTCTGCCTAATAAAAAACATTTA |
| | TTTTCATTGCATTTAAATGTTTAAACAAAAAAAAAAAAAAAAAAAAAAAAAAAAAAAAAAGCAG |
| | GTGGGAGGGTACGTTCGTGATAAACAAACGGCCGCT |
| PL7 | GCCCCTGCAGCCGAATTATATTATTTTTGCCAAATAATTTTTAACAAAAGCTCTGAAGTCTTCTTCA |
| | TTTAAATTCTTAGATGATACTTCATCTGGAAAATTGTCCCAATTAGTAGCATGCCACGCTGTGTAAT |
| | ACGACTCACTATAGGGGGTACCCCTCTCCCTCCCCCCCTAACGTTACTGGCCGAAGCCGCTTGGAA |
| | TAAGGCCGGTGTGCGTTTGTCTATATGTTATTTTCCACCATATTGCCGTCTTTTGGCAATGTGAGGG |
| | CCCGGAAACCTGGCCCTGTCTTCTTGACGAGCATTCCTAGGGGTCTTTCCCCTCTCGCCAAAGGAA |
| | TGCAAGGTCTGTTGAATGTCGTGAAGGAAGCAGTTCCTCTGGAAGCTTCTTGAAGACAAACAACGT |
| | CTGTAGCGACCCTTTGCAGGCAGCGGAACCCCCCACCTGGCGACAGGTGCCTCTGCGGCCAAAAG |
| | CCACGTGTATAAGATACACCTGCAAAGGCGGCACAACCCCAGTGCCACGTTGTGAGTTGGATAGTT |
| | GTGGAAAGAGTCAAATGGCTCTCCTCAAGCGTATTCAACAAGGGGCTGAAGGATGCCCAGAAGGT |
| | ACCCCATTGTATGGGATCTGATCTGGGGCCTCGGTGCACATGCTTTACATGTGTTTAGTCGAGGTTA |
| | AAAAACGTCTAGGCCCCCCGAACCACGGGGACGTGGTTTTCCTTTGAAAAACACGATGATAATAT |
| | GGCCACACATATGGGAGTCAAAGTTCTGTTTGCCCTGATCTGCATCGCTGTGGCCGAGGCCAAGCC |
| | CACCGAGAACAACGAAGACTTCAACATCGTGGCCGTGGCCAGCAACTTCGCGACCACGGATCTCG |
| | ATGCTGACCGCGGGAAGTTGCCCGGCAAGAAGCTGCCGCTGGAGGTGCTCAAAGAGTTGGAAGCC |
| | AATGCCCGGAAAGCTGGCTGCACCAGGGGCTGTCTGATCTGCCTGTCCCACATCAAGTGCACGCCC |
| | AAGATGAAGAAGTTCATCCCAGGACGCTGCCACACCTACGAAGGCGACAAAGAGTCCGCACAGGG |
| | CGGCATAGGCGAGGCGATCGTCGACATTCCTGAGATTCCTGGGTTCAAGGACTTGGAGCCCTTGGA |
| | GCAGTTCATCGCACAGGTCGATCTGTGTGTGGACTGCACAACTGGCTGCCTCAAAGGGCTTGCCAA |
| | CGTGCAGTGTTCTGACCTGCTCAAGAAGTGGCTGCCGCAACGCTGTGCGACCTTTGCCAGCAAGAT |
| | CCAGGGCCAGGTGGACAAGATCAAGGGGGCCGGTGGTGACTAAGGATCCTAGGGCCGCAGCTCG |
| | CTTTCTTGCTGTCCAATTTCTATTAAAGGTTCCTTTGTTCCCTAAGTCCAACTACTAAACTGGGGGA |
| | TATTATGAAGGGCCTTGAGCATTTGGATTCTGCCTAATAAAAAACATTTATTTTCATTGCGTTTAGC |
| | TCGCTTTCTTGCTGTCCAATTTCTATTAAAGGTTCCTTTGTTCCCTAAGTCCAACTACTAAACTGGG |
| | GGATATTATGAAGGGCCTTGAGCATTTGGATTCTGCCTAATAAAAAACATTTATTTTCATTGCATTT |
| | AAATGTTTAAACAAAAAAAAAAAAAAAAAAAAAAAAAAAAAAAAGAGACGGGGTACGTTCGTGAT |
| | ACGGCCGCTTCGAGCAGA |
| PL8 | GCCCCTGCAGCCGAATTATATTATTTTTGCCAAATAATTTTTAACAAAAGCTCTGAAGTCTTCTTCA |
| | TTTAAATTCTTAGATGATACTTCATCTGGAAAATTGTCCCAATTAGTAGCATCACGCTGTGTAATAC |
| | GACTCACTATAGGGGGTACGCCACCATGGGAGTCAAAGTTCTGTTTGCCCTGATCTGCATCGCTGT |
| | GGCCGAGGCCAAGCCCACCGAGAACAACGAAGACTTCAACATCGTGGCCGTGGCCAGCAACTTCG |
| | CGACCACGGATCTCGATGCTGACCGCGGGAAGTTGCCCGGCAAGAAGCTGCCGCTGGAGGTGCTC |
| | AAAGAGTTGGAAGCCAATGCCCGGAAAGCTGGCTGCACCAGGGGCTGTCTGATCTGCCTGTCCCA |
| | CATCAAGTGCACGCCCAAGATGAAGAAGTTCATCCCAGGACGCTGCCACACCTACGAAGGCGACA |
| | AAGAGTCCGCACAGGGCGGCATAGGCGAGGCGATCGTCGACATTCCTGAGATTCCTGGGTTCAAG |
| | GACTTGGAGCCCTTGGAGCAGTTCATCGCACAGGTCGATCTGTGTGTGGACTGCACAACTGGCTGC |
| | CTCAAAGGGCTTGCCAACGTGCAGTGTTCTGACCTGCTCAAGAAGTGGCTGCCGCAACGCTGTGCG |
| | ACCTTTGCCAGCAAGATCCAGGGCCAGGTGGACAAGATCAAGGGGGCCGGTGGTGACTAAGGATC |
| | CTAGGGCCGCAGCTCGCTTTCTTGCTGTCCAATTTCTATTAAAGGTTCCTTTGTTCCCTAAGTCCAA |
| | CTACTAAACTGGGGGATATTATGAAGGGCCTTGAGCATTTGGATTCTGCCTAATAAAAAACATTTA |
| | TTTTCATTGCGTTTAGCTCGCTTTCTTGCTGTCCAATTTCTATTAAAGGTTCCTTTGTTCCCTAAGTC |
| | CAACTACTAAACTGGGGGATATTATGAAGGGCCTTGAGCATTTGGATTCTGCCTAATAAAAAACAT |
| | TTATTTTCATTGCATTTAAATGTTTAAACAAAAAAAAAAAAAAAAAAAAAAAAAAAAAAAAAAG |
| | CAGGTGGGAGGGTACGTTCGTGATAAACAAACGGCCGCT |
| PL9 | GCCCCTGCAGCCGAATTATATTATTTTTGCCAAATAATTTTTAACAAAAGCTCTGAAGTCTTCTTCA |
| | TTTAAATTCTTAGATGATACTTCATCTGGAAAATTGTCCCAATTAGTAGCATGCCACGCTGTGTAAT |
| | ACGACTCACTATAGGGGTAAAACAAAACACACCCCTCTCCCTCCCCCCCTAACGTTACTGGCCGAA |
| | GCCGCTTGGAATAAGGCCGGTGTGCGTTTGTCTATATGTTATTTTCCACCATATTGCCGTCTTTTGG |
| | CAATGTGAGGGCCCGGAAACCTGGCCCTGTCTTCTTGACGAGCATTCCTAGGGGTCTTTCCCCTCTC |
| | GCCAAAGGAATGCAAGGTCTGTTGAATGTCGTGAAGGAAGCAGTTCCTCTGGAAGCTTCTTGAAG |
| | ACAAACAACGTCTGTAGCGACCCTTTGCAGGCAGCGGAACCCCCCACCTGGCGACAGGTGCCTCT |
| | GCGGCCAAAAGCCACGTGTATAAGATACACCTGCAAAGGCGGCACAACCCCAGTGCCACGTTGTG |
| | AGTTGGATAGTTGTGGAAAGAGTCAAATGGCTCTCCTCAAGCGTATTCAACAAGGGGCTGAAGGA |
| | TGCCCAGAAGGTACCCCATTGTATGGGATCTGATCTGGGGCCTCGGTGCACATGCTTTACATGTGT |
| | TTAGTCGAGGTTAAAAAACGTCTAGGCCCCCCGAACCACGGGGACGTGGTTTTCCTTTGAAAAACA |
| | CGATGATAATATGGCCACACATATGGTGTCTAAGGGGGAGGAGCTGTTCACCGGCGTGGTGCCCA |
| | TCCTGGTGGAGCTGGACGGCGACGTGAACGGCCACAAGTTCAGCGTGAGCGGGGAGGGCGAGGG |
| | CGACGCCACCTACGGCAAGCTGACCCTGAAGTTCATCTGCACAACGGGGAAGCTGCCCGTGCCCT |
| | GGCCCACCCTGGTGACCACCCTGACCTACGGCGTGCAGTGCTTCAGCCGGTACCCCGACCACATGA |
| | AGCAGCACGACTTCTTCAAGAGCGCCATGCCCGAGGGGTACGTGCAGGAGCGGACCATCTTCTTC |
| | AAGGACGACGGGAACTACAAGACCCGCGCCGAGGTGAAGTTCGAGGGGGACACCCTGGTGAACC |
| | GCATCGAGCTGAAGGGCATCGACTTCAAGGAGGACGGCAACATCCTGGGGCACAAGCTGGAGTAC |
| | AACTACAACAGCCACAACGTGTACATCATGGCCGACAAGCAGAAGAACGGCATCAAGGTGAACTT |
| | CAAGATCCGGCACAACATCGAGGACGGCAGCGTGCAGCTGGCCGACCACTACCAGCAGAACACCC |
| | CCATCGGCGACGGCCCCGTGCTCCTGCCCGACAACCACTACCTGTCCACCCAGAGCGCCCTGAGCA |
| | AGGACCCAAACGAGAAGCGGGACCACATGGTCCTGCTGGAGTTCGTGACCGCCGCCGGCATCACC |
| | CTGGGCATGGACGAGCTGTACAAGCGCTCCCGCGACATCAGCCACGGCTTCCCTCCCGCCGTGGCC |
| | GCCCAGGACGACGGCACCCTGCCCATGAGCTGCGCCCAGGAGAGCGGCATGGACCGGCACCCGGC |
| | CGCCTGCGCCAGCGCCCGGATCAACGTGTGATAGGGATCCAACAAAAAAAACAAAAAAAAAACC |
| | AAAGAGACGCGGCCGCTTCGAGCAGACATGATAAGATACATT |
| PL10 | GCCCCTGCAGCCGAATTATATTATTTTTGCCAAATAATTTTTAACAAAAGCTCTGAAGTCTTCTTCA |
| | TTTAAATTCTTAGATGATACTTCATCTGGAAAATTGTCCCAATTAGTAGCATGCCACGCTGTGTAAT |
| | ACGACTCACTATAGGGGGAGACCCTCGACCGTCGATTGTCCACTGGTCAACAATAGATGACTTACA |
| | ACTAATCGGAAGGTGCAGAGACTCGACGGGAGCTACCCTAACGTCAAGACGAGGGTAAAGAGAG |
| | AGTCCAATTCTCAAAGCCAATAGGCAGTAGCGAAAGCTGCAAGAGAATGAAAATCCGTTGACCTT |
| | AAACGGTCGTGTGGGTTCAAGTCCCTCCACCCCCACGCCGGAAACGCAATAGCCGAAAAACAAAA |
| | AACAAAAAAACCCCCCTCTCCCTCCCCCCCTAACGTTACTGGCCGAAGCCGCTTGGAATAAGGCCG |
| | GTGTGCGTTTGTCTATATGTTATTTTCCACCATATTGCCGTCTTTTGGCAATGTGAGGGCCCGGAAA |
| | CCTGGCCCTGTCTTCTTGACGAGCATTCCTAGGGGTCTTTCCCCTCTCGCCAAAGGAATGCAAGGTC |
| | TGTTGAATGTCGTGAAGGAAGCAGTTCCTCTGGAAGCTTCTTGAAGACAAACAACGTCTGTAGCGA |
| | CCCTTTGCAGGCAGCGGAACCCCCCACCTGGCGACAGGTGCCTCTGCGGCCAAAAGCCACGTGTAT |
| | AAGATACACCTGCAAAGGCGGCACAACCCCAGTGCCACGTTGTGAGTTGGATAGTTGTGGAAAGA |
| | GTCAAATGGCTCTCCTCAAGCGTATTCAACAAGGGGCTGAAGGATGCCCAGAAGGTACCCCATTGT |
| | ATGGGATCTGATCTGGGGCCTCGGTGCACATGCTTTACATGTGTTTAGTCGAGGTTAAAAAACGTC |
| | TAGGCCCCCCGAACCACGGGGACGTGGTTTTCCTTTGAAAAACACGATGATAATATGGCCACACAT |
| | ATGGGAGTCAAAGTTCTGTTTGCCCTGATCTGCATCGCTGTGGCCGAGGCCAAGCCCACCGAGAAC |
| | AACGAAGACTTCAACATCGTGGCCGTGGCCAGCAACTTCGCGACCACGGATCTCGATGCTGACCG |
| | CGGGAAGTTGCCCGGCAAGAAGCTGCCGCTGGAGGTGCTCAAAGAGTTGGAAGCCAATGCCCGGA |
| | AAGCTGGCTGCACCAGGGGCTGTCTGATCTGCCTGTCCCACATCAAGTGCACGCCCAAGATGAAG |
| | AAGTTCATCCCAGGACGCTGCCACACCTACGAAGGCGACAAAGAGTCCGCACAGGGCGGCATAGG |
| | CGAGGCGATCGTCGACATTCCTGAGATTCCTGGGTTCAAGGACTTGGAGCCCTTGGAGCAGTTCAT |
| | CGCACAGGTCGATCTGTGTGTGGACTGCACAACTGGCTGCCTCAAAGGGCTTGCCAACGTGCAGTG |
| | TTCTGACCTGCTCAAGAAGTGGCTGCCGCAACGCTGTGCGACCTTTGCCAGCAAGATCCAGGGCCA |
| | GGTGGACAAGATCAAGGGGGCCGGTGGTGACTAAAAAAAACAAAAAACAAAACGGCTATTATGC |
| | GTTACCGGCGAGACGCTACGGACTTAAATAATTGAGCCTTAAAGAAGAAATTCTTTAAGTGGATGC |
| | TCTCAAACTCAGGGAAACCTAAATCTAGTTATAGACAAGGCAATCCTGAGCCAAGCCGAAGTAGT |
| | AATTAGTAAGACCAGTGGACAATCGACGGATAACAGCATATCTAGCGGCCGCTTCGAGCAGACAT |
| | GATAAGATACATTGATGATC |

### EXAMPLES

The following examples are provided solely to demonstrate the invention and to explain its aspects. The following examples should not be considered as limiting or demonstrating the full scope of the invention. Examples 1-3 demonstrate the synthesis of representative transcription initiators containing EDA group suitable for the synthesis of pre-circRNAs-starting material for the circularization according to this invention. Example 4 describes the generation of DNA templates for the synthesis of RNAs by transcription in vitro. Examples 5-11 describes the preparation of circRNAs according to the invention as well as reference circRNAs obtained by state-of-the-art methods used in comparative studies. Examples 12-14 describe the procedures for circRNA purification and structure confirmation. Examples 15 and 16 described the optimization of RNA conformation for optimal chemical circularization. Example 17 described the evaluation of translational activity of circ-mRNAs in living cells.

### Example 1. Synthesis of EDA-PEG₅-AG

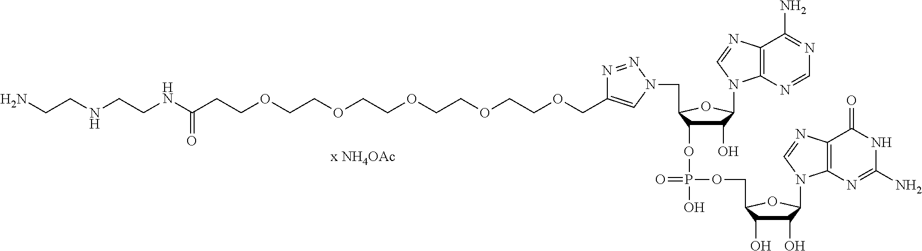

Diethylenetriamine (33.7 μl, 310 μmol, Sigma) was added to a solution of Alkyne-PEG5-NHS ester (12.5 mg, 31.2 μmol, Sigma) in DMF (200 μl). After 150 min of vortexing at 20° C. aqueous solution of ammonium acetate (50 μl, 500 mM, HPLC-grade) and acetic acid (60 μl, 50%) were added to afford a mixture of pH ~7. Next, a solution of N3-AG (28 μmol, 2.00 ml) 31 and a solution of Cu(II)-THPTA complex (60 μl, 50 mM) were combined with the mixture. After that, sodium ascorbate (31 mg, 157 μmol) was added, and the reaction mixture was stirred at 20° C. and monitored by HPLC. After two hours a solution of triethylammonium acetate (200 μl, 1 M, HPLC-grade), a solution of Cu(II)-THPTA complex (20 μl, 50 mM), and sodium ascorbate (114 mg, 586 μmol) were added, and the reaction was continued overnight. The reaction was quenched with EDTA and resolved using HPLC-column: Gemini 5 μm NX-C18 110 Å 250×10 mm; flow 5 ml/min at 23° C.; solvent A: 50 mM NH₄OAc pH 5.9; solvent B: acetonitrile; elution: from 0% to 41% B in 25 min, then from 41% to 100% B in 10 min, then 100% B for 5 min. HPLC eluate containing the desired product was freeze-dried and resuspended in water in three repetitions to remove the traces of solvents and ammonium acetate. The final product EDA-PEG₅-AG was obtained in a form of solution of an ion pair with ammonium acetate (9.2 μmol, 95% purity by HPLC) with 33% yield. MS ESI(−): m/z=1025.8, calculated for C₃₈H₅₈N₁₆O₁₆P m/z: 1025.4.

### Example 2. Synthesis of EDA-AG

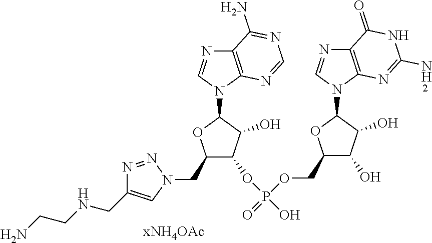 [SMILES: NCCNCc1cn(C[C@H]2O[C@@H](n3cnc4c(N)ncnc43)[C@H](O)[C@@H]2OP(=O)(O)OC[C@H]2O[C@@H](n3cnc4c(=O)[nH]c(N)nc43)[C@H](O)[C@@H]2O)nn1]

Sodium ascorbate (10.3 mg, 52 μmol) and PEDA×2HCl (3.4 mg, 20 μmol) were added to N₃-AG (7.4 mg, 11.5 μmol) 2 and dissolved in degassed triethylamine acetate solution (2.0 mL, 45 mM, pH 7). Then a solution of the CuSO4-THPTA complex (10 μL, 100/500 mM in water) was added and incubated for 2 h at room temperature. Then an EDTA solution (20 μL, 100 mM, pH 7.0) was added and HPLC separation was performed on a C18 column; system A: 100 mM NH₄OAc pH 5.9 B: 30% MeCN, program: 0-25% B in 40 min, 4.5 mL/min 22° C. After combining the fractions and freeze-drying three times, the product was obtained in the form of the ammonium acetate salt (2.8 mg, 3.50 μmol, E₂₆₀=24.3 mM⁻¹cm⁻¹) with a yield of 30%. MS ESI(−): 734.4 (Calc. [M−H]₋: 734.2).

### Example 3. Synthesis of EDA-AG-v2

N₃ApG (42 μmol) was dissolved in MQ water (5.0 ml). To this solution 1M phosphate buffer was added (550 μl, pH=7.0). TCEP hydrochloride (60 mg, 210 μmol was dissolved in 1M NaOH solution (780 μl) and immediately added to N₃-ApG solution. The reaction was monitored by HPLC. After full consumption of the starting material (approximately 2 h) the reaction mixture was freeze-dried. The resulting foam was dissolved in minimal amount of water and the product was isolated by RP flash chromatography (25 g C18 column; Buffer A: 0.05% TFA/water B: MeCN, program: A in 5 column volumes then 0-30 B in 20 column volumes, 40 mL/min). After combining the fractions and freeze-drying three times, the product was obtained in the form of TFA salt (32 μmol) with yield of 76%.

The synthesis of Boc-EDA-ApG NH 2 ApG (9 μmol) was dissolved in 0.1 M AcONa buffer (1.8 ml, pH=5.4) and mixed with 9 μl of N-Boc-2-aminoacetaldehyde solution (1M in DMF) and 40 μl NaBH₃ CN solution (1M in DMF). The reaction was stirred at 45° C. for 3 h (at this point HPLC analysis indicated approximately 25% conversion of the starting material and formation of the product of double reductive amination). The reaction mixture was subjected directly to RP flash chromatography separation (25 g C18 column; Buffer A: 0.05% TFA/water B: MeCN, program: A in 5 column volumes then 0-30 B in 20 column volumes, 40 mL/min). The unreacted starting material was recovered (6 μmol). Combined fractions containing the desired product were freeze-drying three times. The product was obtained in the form of TFA salt (2 μmol) with yield of 22%, MS ESI(−): 753.7 (Calc. [M−H]−: 753.2 for C 27 H 38 N 12 O 12 P—)

The synthesis of EDA-ApG Boc-EDA-ApG (2 μmol) was dissolved in 0.05 M HCl (250 μl) and stirred overnight at 30° C. The reaction mixture was neutralised with 0.1 M NaHCO 3 and subjected directly to RP flash chromatography separation (25 g C18 column; 0.05% TFA/water 5 column volumes 40 mL/min). Combined fractions containing the desired product were freeze-drying three times. The product was obtained in the form of TFA salt (2 μmol) in quantitative yield, MS ESI(−): 653.2 (Calc. [M−H]−: 653.2, for C 22 H 30 N 12 O 10 P—) 1 H NMR (500 MHz, D 2 O) δ 8.13 (s, 1H), 8.12 (s, 1H), 7.92 (s, 1H), 5.93 (d, J=3.4 Hz, 1H), 5.76 (d, J=4.0 Hz, 1H), 4.76-4.73 (m, 1H), 4.59-4.54 (m, 1H), 4.54-4.51 (m, 1H), 4.50-4.45 (m, 1H), 4.44-4.38 (m, 1H), 4.36-4.28 (m, 2H), 4.20-4.10 (m, 1H), 3.22-3.15 (m, 3H), 3.12-3.04 (m, 3H).

### Example 4. DNA Template Preparation

The plasmids were prepared by cloning a sequence of choice (Table 4), containing a T7 RNA polymerase promoter and a site for a specific restrictase cleavage, into a pJet vector. To afford the desired DNA template for IVT the plasmids were linearised with a restriction enzyme according to manufacturer's protocol (Thermo). For details see Table 2.

### Example 5. General Protocol for Preparation of EDA-Linker-RNA (Pre-circRNA 2, 4-6, 8, 10, 12, 13, 15, 17, 21)

| | C_{stock} | V_{stock} [μl] | C_{fin} |
|---|---|---|---|
| H₂O | | up to 50 μl | |
| Transcription Buffer x5 | x5 | 10 | x1 |
| U/A/CTP [mM] | 33.3 | 9 | 6 |
| GTP [mM] | 100 | 2 | 4 |
| EDA-linker-AG | 40 | 15 | 12 |
| [mM] | | | |
| DNA template [ng/μl] | 400 | 5 | 40 |
| MgCl₂ [mM] | 1 000 | 1.25 | 25 |
| Ppase [U/μl] | 0.1 | 1 | 0.002 |
| Ribolock [U/μl] | 40 | 1.25 | 1 |
| T7 RNAP HC [U/μl] | 200 | 5 | 20 |

All ingredients of the reaction mixture listed in the table above were mixed in room temperature in the following order: water, Transcription Buffer ×5 (200 mM Tris-HCl pH 7.9, 30 mM MgCl₂, 50 mM DTT, 50 mM NaCl and 10 mM spermidine, Thermo), NTPs (Thermo), EDA-linker-AG (or EDA-linker-Cap), DNA template, MgCl₂, Ribolock (Thermo), and T7 RNA polymerase HC (Thermo). The reaction mixture was vortexed, gently centrifuged, and incubated for 2 h at 37° C. Next, DNase I (2.0 μl, 1 U/μl, Thermo) was added, and the mixture was incubated for 30 min at 37° C. The RNA product was isolated from the reaction mixture using Monarch RNA cleanup kit (500 μg, NEB). The isolated RNA (~200 μg) was resolved using RP-HPLC—column: PolymerX RP-1 3 μm 100 Å, 4.1×150 mm; flow 1.0 ml/min at 60° C.; solvent A: 100 mM TEAA pH 7.0; solvent B: 200 mM TEAA pH 7.0/acetonitrile 1:1 (v/v); elution: from 20% to 32.5% B in 25 min, then from 32.5% to 100% B in 2 min, then 100% B for 2 min. HPLC eluate containing the desired RNA product was concentrated by precipitation: sodium acetate (0.1 V, 3.0 M, pH 5.2), and isopropanol (1.0-1.5 V) were added to the eluate (initial volume=1 V), and the solution was cooled for 30 min at −80° C. or overnight at −20° C. The pellet was centrifuged (30 min, 14 000 g, 4° C.), washed with 80% ethanol (1.0 ml), centrifuged (10 min, 14 000 g, 4° C.), dried in vacuo, and suspended in water to afford the RNA product (100-180 μg, 50-90% of the crude product).

### Example 6. General Protocol for Preparation of p-RNA (Pre-circRNA 1, 3, 7, 9, 11, 14, 16)

| | C_{stock} | V_{stock} [μl] | C_{fin} |
|---|---|---|---|
| H₂O | | up to 50 μl | |
| Transcription Buffer x5 | x5 | 10 | x1 |
| U/A/CTP [mM] | 33.3 | 9 | 6 |
| GTP [mM] | 100 | 1.5 | 3 |
| GMP [mM] | 100 | 10 | 20 |
| DNA template [ng/μl] | 400 | 5 | 40 |
| MgCl₂ [mM] | 1 000 | 1.25 | 25 |
| Ppase [U/μl] | 0.1 | 1 | 0.002 |
| Ribolock [U/μl] | 40 | 1.25 | 1 |
| T7 RNAP HC [U/μl] | 200 | 5 | 20 |

All ingredients of the reaction mixture listed in the table above were mixed at 37° C. in the following order: water, Transcription Buffer ×5 (200 mM Tris-HCl pH 7.9, 30 mM MgCl₂, 50 mM DTT, 50 mM NaCl and 10 mM spermidine, Thermo), NTPs (Thermo), GMP (preincubated at 37° C.), DNA template, MgCl₂, Ribolock (Thermo), and T7 RNA polymerase HC (Thermo). The reaction mixture was vortexed, gently centrifuged, and incubated for 2 h at 37° C. Next, DNase I (2.0 μl, 1 U/μl, Thermo) was added, and the mixture was incubated for 30 min at 37° C. The crude RNA product was isolated from the reaction mixture using Monarch RNA cleanup kit (500 μg, NEB). The isolated crude RNA (~150 μg, 2.5-3.5 g of RNA per one litter of IVT reaction mixture) was resolved using RP-HPLC—column: PolymerX RP-1 3 μm 100 Å, 4.1×150 mm; flow 1.0 ml/min at 60° C.; solvent A: 100 mM TEAA pH 7.0; solvent B: 200 mM TEAA pH 7.0/acetonitrile 1:1 (v/v); elution: from 20% to 32.5% B in 25 min, then from 32.5% to 100% B in 2 min, then 100% B for 2 min. HPLC eluate containing the desired RNA product was concentrated by precipitation: sodium acetate (0.1 V, 3.0 M, pH 5.2), and isopropanol (1.0-1.5 V) were added to the eluate (initial volume=1 V), and the solution was cooled for 30 min at −80° C. or overnight at −20° C. The pellet was centrifuged (30 min, 14 000 g, 4° C.), washed with 80% ethanol (1.0 ml), centrifuged (10 min, 14 000 g, 4° C.), dried in vacuo, and suspended in water to afford the RNA product (70-130 μg, 50-90% of the crude product).

### Example 7. Chemical Circularization of RNA

A diluted solution of the precursor RNA (30-40 μg in 490 μl, ~140 μM, pre-circRNA 2, 4-6, 8, 10, 12, 13, 15, 17) was mixed with buffer (70 μl, 800 mM KH₂PO₄, 200 mM NaCl, pH 7.0) and incubated at 65° C. for 5 min. After that, the hot solution was transferred on bench, where it cooled to room temperature. After 10-15 min at room temperature, a fresh solution of sodium periodate (70 μl, 10 mM) was added, and the reaction mixture was immediately placed in dark and incubated at 25° C. After 30 min, a fresh solution of sodium cyanoborohydride (70 μl, 200 mM) was added, and the incubation was continued for the next 90 min. The crude circRNA product was isolated from the reaction mixture using Monarch RNA cleanup kit (50 μg, NEB). The isolated RNA (80-99% of the initial amount) was resolved using RP-HPLC—column: PolymerX RP-1 3 μm 100 Å, 4.1×150 mm; flow 1.0 ml/min at 60° C.; solvent A: 100 mM HAA pH 7.0; solvent B: 100 mM HAA pH 7 in 75% acetonitrile; elution: from 55% to 60% B in 25 min, then from 60% to 100% B in 2 min, then 100% B for 2 min. HPLC eluate containing the desired RNA product was concentrated by precipitation: sodium acetate (0.1 V, 3.0 M, pH 5.2), and isopropanol (1.0-1.5 V) were added to the eluate (initial volume=1 V), and the solution was cooled for 30 min at −80° C. or overnight at −20° C. The pellet was centrifuged (30 min, 14 000 g, 4° C.), washed with 80% ethanol (1.0 ml), centrifuged (10 min, 14 000 g, 4° C.), dried in vacuo, and suspended in water to afford the circRNA product (5-10 μg, 15-25% of the initial amount). For additional purification and removal of linear RNA, the product was treated with RNase R as described below (see section RNase R digestion).

### Example 8. Circularization of RNA in the Presence of a Complementary Oligonucleotide

A diluted solution of the precursor RNA (30-40 μg in 480 μl, ~145 μM, pre-circRNA 2, 4-6, 8, 10, 12, 13, 15, 17) was mixed with buffer (70 μl, 800 mM KH₂PO₄, 200 mM NaCl, pH 7.0), and complementary oligonucleotide (~11 μl, 10 μM, 1.5 eq, see Table 3 for sequence) and incubated at 65° C. for 5 min. After that, the hot solution was transferred on bench, where it cooled to room temperature. After 10-15 min at room temperature, a fresh solution of sodium periodate (70 μl, 10 mM) was added, and the reaction mixture was immediately placed in the dark and incubated at 25° C. After 30 min, a fresh solution of sodium cyanoborohydride (70 μl, 200 mM) was added, and the incubation was continued for the next 90 min. The crude circRNA product was isolated from the reaction mixture using Monarch RNA cleanup kit (50 μg, NEB). The isolated RNA was resolved using RP-HPLC—column: PolymerX RP-1 3 μm 100 Å, 4.1×150 mm; flow 1.0 ml/min at 60° C.; solvent A: 100 mM HAA pH 7.0; solvent B: 100 mM HAA pH 7 in 75% acetonitrile; elution: from 55% to 60% B in 25 min, then from 60% to 100% B in 2 min, then 100% B for 2 min. HPLC eluate containing the desired RNA product was concentrated by precipitation: sodium acetate (0.1 V, 3.0 M, pH 5.2), and isopropanol (1.0-1.5 V) were added to the eluate (initial volume=1 V), and the solution was cooled for 30 min at −80° C. or overnight at −20° C. The pellet was centrifuged (30 min, 14 000 g, 4° C.), washed with 80% ethanol (1.0 ml), centrifuged (10 min, 14 000 g, 4° C.), dried in vacuo, and suspended in water to afford the circRNA product (5-10 μg, 15-25% of the initial amount). For additional purification and removal of linear RNA, the product was treated with RNase R as described below (see section RNase R digestion).

### Example 9. Enzymatic Circularization with T4 RNA Ligase I

A diluted solution of the precursor RNA (10-15 μg in 95 μl, ~150 μM, pre-circRNA 1, 3, 7, 9, 11, 14, 16) was mixed with buffer (5 μl, 100 mM Tris-HCl, 500 mM NaCl, pH 7.0) and incubated at 65° C. for 5 min. After that, the hot solution was transferred on bench, where it cooled to room temperature. After 10-15 min at room temperature, T4 RNA ligase 1 buffer ×10 (15 μl, 500 mM Tris-HCl, 100 mM MgCl₂, 10 mM DTT, pH 7.5, NEB), PEG 8000 (15 μl, 50%, NEB), ATP (0.75 μl, 10 mM, NEB), RiboLock RNase inhibitor (3.75 μl, 40 U/μl, Thermo), and T4 RNA ligase 1 (15 μl, 10 U/μl, NEB) were added, and the reaction mixture was incubated at 25° C. for 150 min. The crude circRNA product was isolated from the reaction mixture using Monarch RNA cleanup kit (10 μg, NEB). The isolated RNA (80-99% of the initial amount) was resolved using RP-HPLC—column: RNASept Prep 2 μm 100 Å, 7.8×50 mm; flow 1.0 ml/min at 60° C.; solvent A: 100 mM HAA pH 7.0 in 10% acetonitrile; solvent B: 100 mM HAA pH 7 in 75% acetonitrile; elution: from 20% to 70% B in 25 min, then from 70% to 100% B in 2 min, then 100% B for 2 min. HPLC eluate containing the desired RNA product was concentrated by precipitation: sodium acetate (0.1 V, 3.0 M, pH 5.2), and isopropanol (1.0-1.5 V) were added to the eluate (initial volume=1 V), and the solution was cooled for 30 min at −80° C. or overnight at −20° C. The pellet was centrifuged (30 min, 14 000 g, 4° C.), washed with 80% ethanol (1.0 ml), centrifuged (10 min, 14 000 g, 4° C.), dried in vacuo, and suspended in water to afford the circRNA product (1-4 μg, 10-30% of the crude product). For additional purification and removal of linear RNA, the product was treated with RNase R as described below (see section RNase R digestion).

### Example 10. Enzymatic Circularization with T4 RNA Ligase II

A diluted solution of the precursor RNA (30-40 μg in 220 μl, ~320 μM, pre-circRNA 1, 3, 7, 9, 11, 14, 16) was mixed with complementary oligonucleotide (~11 μl, 10 μM, 1.5 eq, see Table 3 for sequence) and incubated at 65° C. for 5 min. After that, the hot solution was transferred on bench, where it cooled to room temperature. After 10-15 min at room temperature, T4 RNA ligase 2 buffer ×10 (35 μl, 500 mM Tris-HCl, 20 mM MgCl₂, 10 mM DTT, 4 mM ATP, pH 7.5, NEB), PEG 8000 (70 μl, 50%, NEB), RiboLock RNase inhibitor (8.75 μl, 40 U/μl, Thermo), and T4 RNA ligase 2 (3.5 μl, 10 U/μl, NEB) were added, and the reaction mixture was incubated at 25° C. for 150 min. The crude circRNA product was isolated from the reaction mixture using Monarch RNA cleanup kit (500 μg, NEB). The isolated RNA (80-99% of the initial amount) was resolved using RP-HPLC—column PolymerX RP-1 3 μm 100 Å, 4.1×150 mm; flow 1.0 ml/min at 60° C.; solvent A: 100 mM HAA pH 7.0; solvent B: 100 mM HAA pH 7 in 75% acetonitrile; elution: from 55% to 60% B in 25 min, then from 60% to 100% B in 2 min, then 100% B for 2 min. HPLC eluate containing the desired RNA product was concentrated by precipitation: sodium acetate (0.1 V, 3.0 M, pH 5.2), and isopropanol (1.0-1.5 V) were added to the eluate (initial volume=1 V), and the solution was cooled for 30 min at −80° C. or overnight at −20° C. The pellet was centrifuged (30 min, 14 000 g, 4° C.), washed with 80% ethanol (1.0 ml), centrifuged (10 min, 14 000 g, 4° C.), dried in vacuo, and suspended in water to afford the circRNA product (5-10 μg, 15-25% of the initial amount). For additional purification and removal of linear RNA, the product was treated with RNase R as described below (see section RNase R digestion).

### Example 11. Self-Splicing Circularization Using Permuted Intron-Exon (PIE) System

| | C_{stock} | V_{stock} [μl] | C_{fin} |
|---|---|---|---|
| H₂O | | up to 20 μl | |
| Transcription Buffer x5 | x5 | 4 | x1 |
| U/A/CTP [mM] | 33.3 | 3 | 5 |
| GTP [mM] | 100 | 1 | 5 |
| DNA template [ng/μl] | 400 | 5 | 40 |
| MgCl₂ [mM] | 1 000 | 0.4 | 20 |
| Ppase [U/μl] | 0.1 | 0.4 | 0.002 |
| Ribolock [U/μl] | 40 | 0.5 | 1 |
| T7 RNAP HC [U/μl] | 200 | 1 | 10 |

Linear precursor and circular product of mRNA (pre-circRNA18 and circRNA18) were produced by in vitro transcription from a linearized plasmid DNA (PL10). All ingredients of the reaction mixture listed in the table above were mixed in room temperature in the following order: water, Transcription Buffer ×5 (200 mM Tris-HCl pH 7.9, 30 mM MgCl₂, 50 mM DTT, 50 mM NaCl and 10 mM spermidine, Thermo), NTPs (Thermo), DNA template, MgCl₂, Ribolock (Thermo), and T7 RNA polymerase HC (Thermo). The reaction mixture was vortexed, gently centrifuged, and incubated for 2 h at 37° C. Next, DNase I (2.0 μl, 1 U/μl, Thermo) was added, and the mixture was incubated for 30 min at 37° C. The RNA product was isolated from the reaction mixture using Monarch RNA cleanup kit (500 μg, NEB). The isolated RNA (~200 μg) was resolved using RP-HPLC—column: PolymerX RP-1 3 μm 100 Å, 4.1×150 mm; flow 1.0 ml/min at 60° C.; solvent A: 100 mM TEAA pH 7.0; solvent B: 200 mM TEAA pH 7.0/acetonitrile 1:1 (v/v); elution: from 20% to 32.5% B in 25 min, then from 32.5% to 100% B in 2 min, then 100% B for 2 min. HPLC eluate containing the desired RNA product was concentrated by precipitation: sodium acetate (0.1 V, 3.0 M, pH 5.2), and isopropanol (1.0-1.5 V) were added to the eluate (initial volume=1 V), and the solution was cooled for 30 min at −80° C. or overnight at −20° C. The pellet was centrifuged (30 min, 14 000 g, 4° C.), washed with 80% ethanol (1.0 ml), centrifuged (10 min, 14 000 g, 4° C.), dried in vacuo, and suspended in water to afford the RNA product (100-180 μg, 50-90% of the crude product). For circRNA, additional GTP was added to final concentration of 2 mM along with a buffer including magnesium. RNA was heated to 55° C. for 8 min, and then column purified.

### Example 12. RP-HPLC of circRNA

All HPLC separations were performed on modular, low-pressure gradient HPLC apparatus manufactured by Shimadzu (LC-40/LC-20 series) with a thermostated column holder or column oven, DAD detector and fluorescence detector. The mobile phase was composed of non-autoclaved MQ water, HPLC-grade acetonitrile (J.T.Baker), high quality triethylamine (Bioultra, Sigma), HPLC-grade n-hexylamine (Ottokemi), and HPLC-grade glacial acetic acid (J.T.Baker). Solvents of desired composition, concentration and pH were filtered with 0.45 μm filter and stored in the dark at 4° C. for longer periods of time. Chromatograms were recorded with the detection of absorbance at 260 nm (ref 360 nm, 10 nm bandwidth), absorbance spectrum (220-700 nm), and fluorescence of interest (Cy3-550/565, Cy5-650/665).

### Example 13. RNase R Digestion

A solution of RNA (43 μl, 10 μg) was incubated at 65° C. for 5 min and rapidly cooled in an ice bath. After 5 min at 0-4° C., RNase R buffer ×10 (5 μl, 200 mM Tris-HCl, 1 M KCl, 1 mM MgCl₂, pH 7.5, ABM), RiboLock RNase inhibitor (1.25 μl, 40 U/μl, Thermo), and RNase R (1 μl, 10 U/μl, ABM) were added. After 30 min of incubation at 37° C. the products of digestion were analyzed with PAGE directly or isolated from the reaction mixture using Monarch RNA cleanup kit (10 μg, NEB).

### Example 14. RNase H Assay

A solution (8.5 μl) containing circRNA2 (100 ng), DNA oligonucleotide (0 or 150 nM, CCTTCAGCTCGATGCGGTTCA), and RNase H buffer ×1 (20 mM Tris-HCl, 10 mM KCl, 8 mM MgCl₂, 1 mM DTT, Thermo) was heated to 65° C. for 5 min and gradually cooled to 37° C. for 30 min. Next, RiboLock RNase inhibitor (0.5 μl, 40 U/μl, Thermo), and RNase H (1 μl, 5 U/μl, Thermo) were added. After 30 min of incubation at 37° C. the products of digestion were directly analyzed with PAGE.

### Example 15. FRET Probe Synthesis

The Cy5-RNA1-Cy3 FRET probe was synthesized according to the previously reported protocol.³¹ First in vitro transcription of RNA1 sequence was performed in the presence of N3-AG transcription initiator. The crude product of IVT was isolated by Monarch RNA cleanup kit (NEB) and subjected to 5′-Cy5 and 3′-Cy3 dual labeling. The crude labeling product was isolated from the labeling reaction mixture by Monarch RNA cleanup kit (NEB) and subsequently resolved with HPLC. The HPLC eluate containing the desired product was concentrated by precipitation dried in vacuo and suspended in water to afford the Cy5-RNA1-Cy3 FRET probe solution.

### Example 16. FRET Experiments

The Cy5-RNA1-Cy3 FRET probe solution was diluted with buffer (80 mM KH₂PO₄, 20 mM NaCl, pH 7.0, final volume of 100 μl) to afford a final probe concentration of 100 nM and transferred to a quartz fluorescence cuvette (1×1×350 mm). Optionally, a solution of a complementary DNA oligonucleotide (2 μl, 10 μM, ON2-ON5) was added. The mixture was heated from 20 to 90° C. in 8 min and gradually cooled to 20° C. or 4° C. in 20-30 min. Emission spectra were recorded on a Cary Eclipse spectrofluorometer (Agilent), equipped with a xenon lamp, single cell peltier accessory (spvf 1x0, Agilent), excitation at 500 nm, emission 510-800 nm, 10 mm slit.

### Example 17. Cell Culture and Transfections

The analysis was performed in four cell lines, i.e. HEK 293T, Hep G2, A549, and HeLa, as well as in mouse dendritic cells and macrophages established from bone marrow monocytes. One day before transfection, 8×10³ cells were seeded per well of a 96-well plate. The next day, cells were transfected with RNA. In each cell line, each RNA variant was transfected in triplicate. Transfection was performed with Lipofectamine Messenger MAX (Invitrogen, Waltham, MA, USA), according to the manufacturer's instructions. During transfection, the molar concentration of each RNA was taken into account so that an equal amount of RNA molecules was added to each well. Briefly, for each reaction 0.15 μL of Lipofectamine was first diluted (Mix 1) in 5 μL of Opti-MEM (GIBCO, Grand Island, NY, USA) medium and incubated at room temperature for 10 minutes. In another tube, the RNA was diluted in 5 μL of Opti-MEM medium (Mix 2). Next, both Mix 1 and Mix 2 were mixed and incubated for 5 minutes at room temperature. Afterward, 10 μL of the mixture was transferred to the appropriate well of a 96-well plate containing previously seeded cells. After the transfection, the cells were incubated in standard culture conditions (37° C., 5% CO₂, saturating humidity). At appropriate time points, 20 μL of medium from each well was withdrawn to test the amount of produced protein, and the remaining medium was completely replaced with the fresh one. The amount of luciferase released into the medium was analyzed using the GLuc GLOW Assay (NanoLight Technologies, Norman, OK, USA) reagents, according to the manufacturer's instructions. Luminescence was read in white, opaque plates. Measurements were made using an En Vision plate reader (Perkin Elmer, Waltham, MA, USA).

### CONCLUSIONS

To summarize the presented results, it should be stated that they confirm unexpected advantages of the chemical cyclization proposed according to the invention compared to known methods using enzymatic ligation.

The method according to the invention allows to achieve better cyclization efficiency.

Moreover, the method according to the invention allows to reduce the formation of unfavorable by-products (such as concatemers, dimers), which contaminate the product obtained by known enzymatic methods.

Further, the method of the invention is independent of the sequence and length of the mRNA being circularized. In particular, circularized mRNA may contain sequences such as: 5′ and 3′ UTR, CDS, poly(A).

The method of the invention is also compatible with possible other modifications occurring within the polynucleotide chain (such as, for example, N-methylpseudouridine or the presence of detectable tags or labels).

Moreover, the method of the invention is more advantageous than the known PIE method. This is evidenced by the profile of by-products formed in both reactions.

### REFERENCES

(1) Qin, S.; Tang, X.; Chen, Y.; Chen, K.; Fan, N.; Xiao, W.; Zheng, Q.; Li, G.; Teng, Y.; Wu, M.; et al. mRNA-based therapeutics: powerful and versatile tools to combat diseases. Signal Transduction and Targeted Therapy 2022, 7 (1). DOI: 10.1038/s41392-022-01007-w (accessed 2022 Oct. 7 T12:03:06). Janowski, M.; Andrzejewska, A. The legacy of mRNA engineering: A lineup of pioneers for the Nobel Prize. Molecular Therapy—Nucleic Acids 2022, 29, 272-284. DOI: 10.1016/j.omtn.2022.07.003 (accessed 2022 Oct. 7 T11:43:22).
(2) Garneau, N. L.; Wilusz, J.; Wilusz, C. J. The highways and byways of mRNA decay. Nature Reviews Molecular Cell Biology 2007, 8 (2), 113-126. DOI: 10.1038/nrm2104. Houseley, J.; Tollervey, D. The Many Pathways of RNA Degradation. Cell 2009, 136 (4), 763-776. DOI: 10.1016/j.cell.2009.01.019 (accessed 2022 Oct. 7 T12:34:14).
(3) Jeck, W. R.; Sharpless, N. E. Detecting and characterizing circular RNAs. Nature Biotechnology 2014, 32 (5), 453-461. DOI: 10.1038/nbt.2890 (accessed 2022 Oct. 7 T12:45:35).
(4) Dolinnaya, N. G.; Blumenfeld, M.; Merenkova, I. N.; Oretskaya, T. S.; Krynetskaya, N.; Ivanovskaya, M. G.; Vasseur, M.; Shabarova, Z. A. Oligonucleotide circularization by template-directed chemical ligation. Nucleic Acids Research 1993, 21 (23), 5403-5407. DOI: 10.1093/nar/21.23.5403 (accessed Oct. 7, 2022). Fedorova, O. A.; Gottikh, M. B.; Oretskaya, T. S.; Shabarova, Z. A. Cyanogen Bromide-Induced Chemical Ligation: Mechanism and Optimization of the Reaction Conditions. Nucleosides and Nucleotides 1996, 15 (6), 1137-1147. DOI: 10.1080/07328319608007382.
(5) Nakamoto, K.; Abe, N.; Tsuji, G.; Kimura, Y.; Tomoike, F.; Shimizu, Y.; Abe, H. Chemically synthesized circular RNAs with phosphoramidate linkages enable rolling circle translation. Chemical Communications 2020, 56 (46), 6217-6220, 10.1039/DOCC02140G. DOI: 10.1039/DOCC02140G.
(6) Rigden, J. E.; Rezaian, M. A. In Vitro synthesis of an infectious viroid: Analysis of the infectivity of monomeric linear CEV. Virology 1992, 186 (1), 201-206. DOI: https://doi.org/10.1016/0042-6822(92)90074-Y.
(7) Chen, C.-y.; Sarnow, P. Initiation of Protein Synthesis by the Eukaryotic Translational Apparatus on Circular RNAs. Science 1995, 268 (5209), 415-417. DOI: 10.1126/science.7536344 (accessed 2022 Oct. 7).
(8) Litke, J. L.; Jaffrey, S. R. Highly efficient expression of circular RNA aptamers in cells using autocatalytic transcripts. Nature Biotechnology 2019, 37 (6), 667-675. DOI: 10.1038/s41587-019-0090-6 (accessed 2022 Oct. 7 T13:55:58).
(9) Kumar, A.; Palmer, N.; Miyasaki, K.; Finburgh, E.; Xiang, Y.; Portell, A.; Dailamy, A.; Suhardjo, A.; Chew, W. L.; Kwon, E. J.; et al. Extensive <i>in vitro</i> and <i>in vivo</i> protein translation via <i>in situ</i> circularized RNAs. Cold Spring Harbor Laboratory: 2022.
(10) Ford, E.; Ares, M. Synthesis of circular RNA in bacteria and yeast using RNA cyclase ribozymes derived from a group I intron of phage T4. Proceedings of the National Academy of Sciences 1994, 91 (8), 3117-3121. DOI: 10.1073/pnas.91.8.3117 (accessed 2022 Oct. 7 T13:11:48).
(11) Wesselhoeft, R. A.; Kowalski, P. S.; Anderson, D. G. Engineering circular RNA for potent and stable translation in eukaryotic cells. Nature Communications 2018, 9 (1). DOI: 10.1038/s41467-018-05096-6 (accessed 2022 Oct. 7 T13:35:12).
(12) Wesselhoeft, R. A.; Kowalski, P. S.; Parker-Hale, F. C.; Huang, Y.; Bisaria, N.; Anderson, D. G. RNA Circularization Diminishes Immunogenicity and Can Extend Translation Duration In Vivo. Molecular Cell 2019, 74 (3), 508-520.e504. DOI: 10.1016/j.molcel.2019.02.015 (accessed 2022 Oct. 7 T13:26:51).
(13) Ni, L.; Yamada, T.; Murata, A.; Nakatani, K. Mismatch binding ligand upregulated back-splicing reaction producing circular RNA in a cellular model. Chemical Communications 2022, 58 (22), 3629-3632. DOI: 10.1039/dlcc06936e (accessed 2022 Oct. 7 T13:11:41).
(14) Qu, L.; Yi, Z.; Shen, Y.; Lin, L.; Chen, F.; Xu, Y.; Wu, Z.; Tang, H.; Zhang, X.; Tian, F.; et al. Circular RNA vaccines against SARS-COV-2 and emerging variants. Cell 2022, 185 (10), 1728-1744.e1716. DOI: 10.1016/j.cell.2022.03.044 (accessed 2022 Oct. 7 T13:26:16).
(15) Nichols, N. M.; Tabor, S.; McReynolds, L. A. RNA Ligases. Current Protocols in Molecular Biology 2008, 84 (1), 3.15.11-13.15.14, https://doi.org/10.1002/0471142727.mb0315s84. DOI: https://doi.org/10.1002/0471142727.mb0315s84 (accessed 2022 Oct. 7).
(16) Kaufmann, G.; Klein, T.; Littauer, U. Z. T4 RNA ligase: Substrate chain length requirements. FEBS Letters 1974, 46 (1-2), 271-275. DOI: 10.1016/0014-5793 (74) 80385-6 (accessed 2022 Oct. 7 T13:11:45).
(17) Puttaraju, M.; Been, M. Group I permuted intron-exon (PIE) sequences self-splice to produce circular exons. Nucleic Acids Research 1992, 20 (20), 5357-5364. DOI: 10.1093/nar/20.20.5357 (accessed 2022 Oct. 10 T16:55:04).
(18) Ramanathan, A.; Robb, G. B.; Chan, S.-H. mRNA capping: biological functions and applications. Nucleic Acids Research 2016, 44 (16), 7511-7526. DOI: 10.1093/nar/gkw551 (accessed Oct. 7, 2022). Topisirovic, I.; Svitkin Yuri, V.; Sonenberg, N.; Shatkin Aaron, J. Cap and cap-binding proteins in the control of gene expression. Wiley Interdisciplinary Reviews: RNA 2010, 2 (2), 277-298. DOI: 10.1002/wrna.52 (accessed 2018 May 6). Mitchell, S. F.; Walker, S. E.; Algire, M. A.; Park, E.-H.; Hinnebusch, A. G.; Lorsch, J. R. The 5′-7-Methylguanosine Cap on Eukaryotic mRNAs Serves Both to Stimulate Canonical Translation Initiation and to Block an Alternative Pathway. Molecular Cell 2010, 39 (6), 950-962. DOI: 10.1016/j.molcel.2010.08.021 (accessed 2022 Oct. 7 T14:25:08).
(19) Koch, A.; Aguilera, L.; Morisaki, T.; Munsky, B.; Stasevich, T. J. Quantifying the dynamics of IRES and cap translation with single-molecule resolution in live cells. Nature Structural & Molecular Biology 2020, 27 (12), 1095-1104. DOI: 10.1038/s41594-020-0504-7.
(20) Warminski, M.; Sikorski, P. J.; Kowalska, J.; Jemielity, J. Applications of Phosphate Modification and Labeling to Study (m) RNA Caps. Topics in Current Chemistry 2017, 375 (1), 16. DOI: 10.1007/s41061-017-0106-y.
(21) Sikorski, P. J.; Warminski, M.; Kubacka, D.; Ratajczak, T.; Nowis, D.; Kowalska, J.; Jemielity, J. The identity and methylation status of the first transcribed nucleotide in eukaryotic mRNA 5′ cap modulates protein expression in living cells. Nucleic acids research 2020, 48 (4), 1607-1626.
(22) Chen, C.-K.; Cheng, R.; Demeter, J.; Chen, J.; Weingarten-Gabbay, S.; Jiang, L.; Snyder, M. P.; Weissman, J. S.; Segal, E.; Jackson, P. K.; et al. Structured elements drive extensive circular RNA translation. Molecular Cell 2021, 81 (20), 4300-4318.e4313. DOI: 10.1016/j.molcel.2021.07.042 (accessed 2022 Oct. 7). Yang, Y.; Fan, X.; Mao, M.; Song, X.; Wu, P.; Zhang, Y.; Jin, Y.; Yang, Y.; Chen, L.-L.; Wang, Y.; et al. Extensive translation of circular RNAs driven by N6-methyladenosine. Cell Research 2017, 27 (5), 626-641. DOI: 10.1038/cr.2017.31 (accessed 2022 Oct. 7 T13:35:12). Fan, X.; Yang, Y.; Chen, C.; Wang, Z. Pervasive translation of circular RNAs driven by short IRES-like elements. Nature Communications 2022, 13 (1). DOI: 10.1038/s41467-022-31327-y (accessed 2022 Oct. 10 T11:25:34).
(23) Lei, M.; Zheng, G.; Ning, Q.; Zheng, J.; Dong, D. Translation and functional roles of circular RNAs in human cancer. Molecular Cancer 2020, 19 (1). DOI: 10.1186/s12943-020-1135-7 (accessed 2022 Oct. 7 T13:34:49). Prats, A.-C.; David, F.; Diallo, L. H.; Roussel, E.; Tatin, F.; Garmy-Susini, B.; Lacazette, E. Circular RNA, the Key for Translation. International Journal of Molecular Sciences 2020, 21 (22), 8591. DOI: 10.3390/ijms21228591 (accessed 2022 Oct. 7 T13:30:21).
(24) Martinez-Salas, E.; Francisco-Velilla, R.; Fernandez-Chamorro, J.; Embarek, A. M. Insights into Structural and Mechanistic Features of Viral IRES Elements. Frontiers in Microbiology 2018, 8, Review. Marques, R.; Lacerda, R.; Romão, L. Internal Ribosome Entry Site (IRES)-Mediated Translation and Its Potential for Novel mRNA-Based Therapy Development. Biomedicines 2022, 10 (8), 1865. DOI: 10.3390/biomedicines10081865 (accessed 2023 Feb. 13 T14:08:04). Chen, R.; Wang, S. K.; Belk, J. A.; Amaya, L.; Li, Z.; Cardenas, A.; Abe, B. T.; Chen, C.-K.; Wender, P. A.; Chang, H. Y. Engineering circular RNA for enhanced protein production. Nature Biotechnology 2022. DOI: 10.1038/s41587-022-01393-0 (accessed 2022 Oct. 7 T11:58:17).
(25) Nowotny, M.; Gaidamakov, S. A.; Ghirlando, R.; Cerritelli, S. M.; Crouch, R. J.; Yang, W. Structure of human RNase H1 complexed with an RNA/DNA hybrid: insight into HIV reverse transcription. Molecular cell 2007, 28 (2), 264-276.
(26) Jacques, J.-P.; Susskind, M. M. Use of electrophoretic mobility to determine the secondary structure of a small antisense RNA. Nucleic Acids Research 1991, 19 (11), 2971-2977. DOI: 10.1093/nar/19.11.2971 (accessed 2022 Oct. 7 T16:18:29). Cebrián, J.; Kadomatsu-Hermosa, M. J.; Castán, A.; Martínez, V.; Parra, C.; Fernández-Nestosa, M. J.; Schaerer, C.; Martínez-Robles, M.-L.; Hernández, P.; Krimer, D. B.; et al. Electrophoretic mobility of supercoiled, catenated and knotted DNA molecules. Nucleic Acids Research 2015, 43 (4), e24-e24. DOI: 10.1093/nar/gku1255 (accessed Oct. 7, 2022).
(27) Abe, B. T.; Wesselhoeft, R. A.; Chen, R.; Anderson, D. G.; Chang, H. Y. Circular RNA migration in agarose gel electrophoresis. Molecular Cell 2022, 82 (9), 1768-1777.e1763. DOI: 10.1016/j.molcel.2022.03.008 (accessed 2022 Oct. 7 T13:26:14).
(28) Haque, F. M.; Grayson, S. M. The synthesis, properties and potential applications of cyclic polymers. Nature Chemistry 2020, 12 (5), 433-444. DOI: 10.1038/s41557-020-0440-5 (accessed 2022 Oct. 7 T13:56:08).
(29) Vicens, Q.; Kieft, J. S. Thoughts on how to think (and talk) about RNA structure. Proceedings of the National Academy of Sciences 2022, 119 (17). DOI: 10.1073/pnas.2112677119 (accessed 2022 Oct. 7 T12:03:32).
(30) Kibbe, W. A. OligoCalc: an online oligonucleotide properties calculator. Nucleic Acids Research 2007, 35 (suppl_2), W43-W46. DOI: 10.1093/nar/gkm234 (accessed Oct. 10, 2022).
(31) Mamot, A.; Sikorski, P. J.; Siekierska, A.; de Witte, P.; Kowalska, J.; Jemielity, J. Ethylenediamine derivatives efficiently react with oxidized RNA 3′ ends providing access to mono and dually labelled RNA probes for enzymatic assays and in vivo translation. Nucleic Acids Research 2022, 50 (1), e3-e3. DOI: 10.1093/nar/gkab867 (accessed Feb. 17, 2022).

1. A circRNA analog according to formula 1 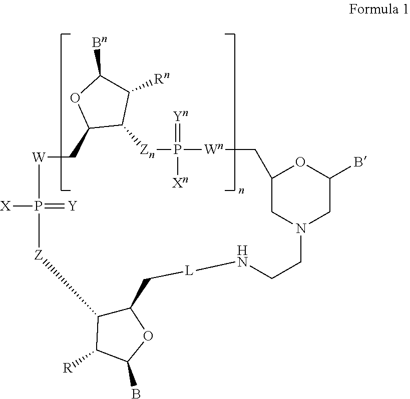
wherein:
n is any integer with a value from 1 to 20 000;
X and each of Xⁿ is independently OH, BH₃, NH₂, or SH;
Y and each of Yⁿ is independently O or S;
Z and each of Zⁿ is independently O, S or NH;
W and each of Wⁿ is independently O, S or NH;
R and each of Rⁿ is independently H, OH, alkyl, O-alkyl or halogen;
B, B′ and each of Bⁿ is independently a natural, modified or unnatural nucleoside base;
L is a linear or branched linker.

2. A circRNA analog according to claim 1, wherein
the linker L is a single bond or a linker according to one among of formulas 2-7:

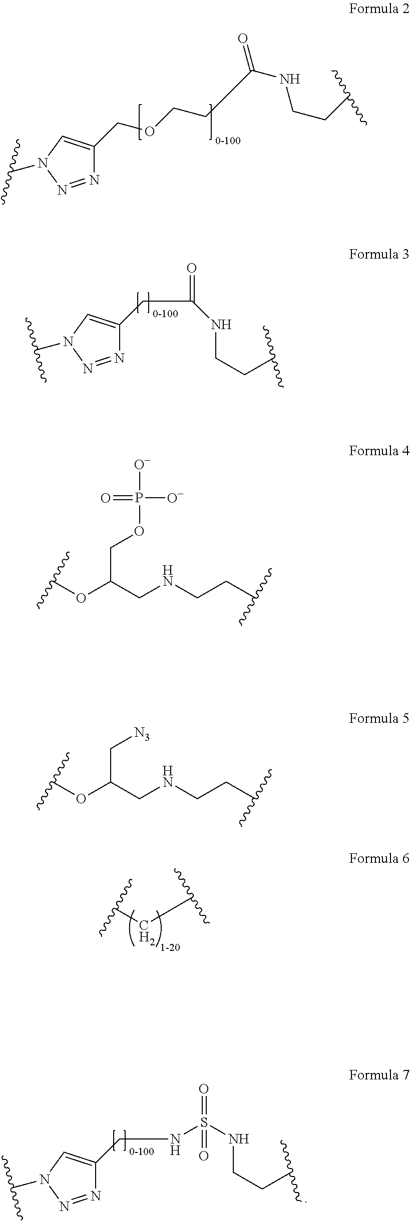

3. A circRNA analog according to any of claims 1-2, wherein:
the linker L comprises a detectable tag or label.

4. A method of synthesis of circRNA analogs described in any of claims 1-3, characterized by that a circRNA analog precursor (formula 8) 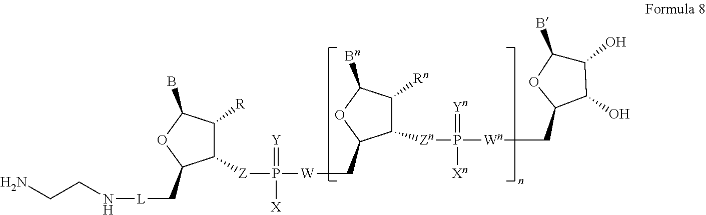
wherein:
n=1-20 000;
X and each of Xⁿ is independently OH, BH₃, NH₂, or SH;
Y and each of Yⁿ is independently O or S;
Z and each of Zⁿ is independently O, S or NH;
W and each of Wⁿ is independently O, S or NH;
R and each of Rⁿ is independently H, OH, alkyl, O-alkyl or halogen;
B, B′ and each of Bⁿ is independently a natural, modified or unnatural nucleoside base;
L is a linear or branched linker;
is first oxidized during step (i) of the method, yielding a dialdehyde derivative (formula 9), 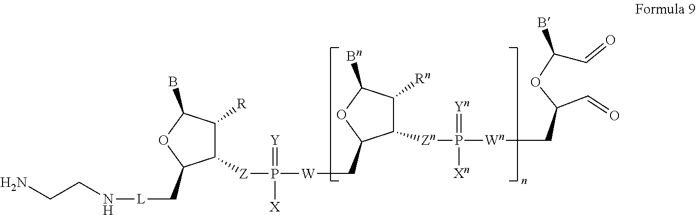
wherein:
n=1-20 000;
X and each of Xⁿ is independently OH, BH₃, NH₂, or SH;
Y and each of Yⁿ is independently O or S;
Z and each of Zⁿ is independently O, S or NH;
W and each of Wⁿ is independently O, S or NH;
R and each of Rⁿ is independently H, OH, alkyl, O-alkyl or halogen;
B, B′ and each of Bⁿ is independently a natural, modified or unnatural nucleoside base;
L is a linear or branched linker;
which is next incubated in reductive environment during step (ii) of the method, yielding the desired circRNA analog.

5. A method according to claim 4, wherein
the linker L is a single bond or a linker according to one among of formulas 2-7:

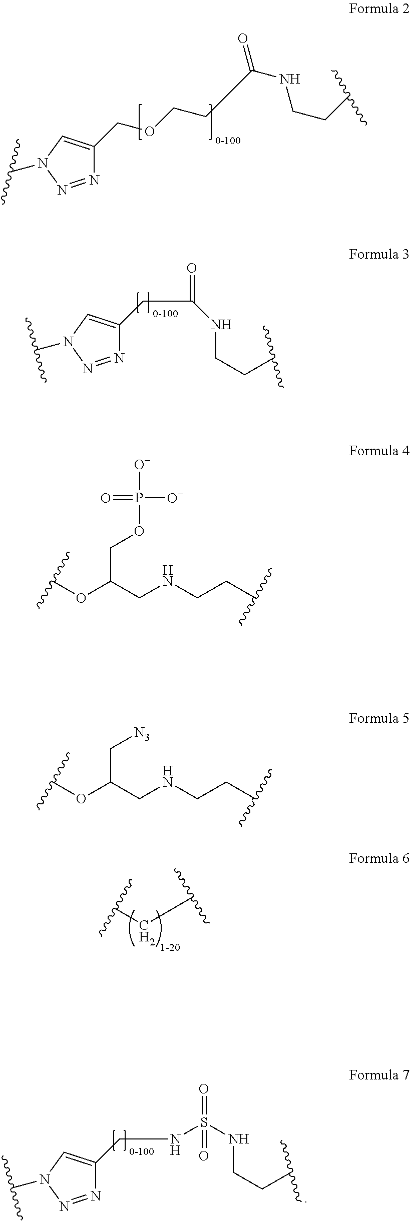

6. A method according to claim 4, characterized by that step (i) and step (ii) are carried out in one reactor.

7. A method according to claim 4, characterized by that step (i) of the method is carried out in the presence of NaIO₄, preferably at a concentration below 10 mM, at a temperature below 60° C.

8. A method according to claim 4, characterized by that step (ii) of the method is carried out in buffered solution, preferably at pH 5-8, in presence of NaBH₃CN below 100 mM.

9. A method according to claim 4, characterized by that step (i) or step (ii) of the method are carried out in presence of a natural, modified or unnatural nucleic acid oligomer that is complementary to a RNA precursor sequence.

10. A method according to claim 4, characterized by that the circRNA analog is isolated from the reaction mixture by a known method of RNA isolation, such as precipitation in alcohol, size exclusion chromatography, gel electrophoresis, or high-performance liquid chromatography (HPLC).

11. Use of a circRNA analog according to any of claims 1-3 in protein production in an in vitro setup, which includes but is not limited to artificial reaction mixture, cellular lysates, nuclear lysates, cell cultures, and tissue cultures.

12. A circRNA analog according to any of claims 1-3 for use in therapeutic or diagnostic method, in particular for protein production in an in vivo setup, which includes but is not limited to whole organisms, organs, cellular and tissue transplants.

13. A pharmaceutical composition comprising of a circRNA analog according to any of claims 1-3.
